(19) United States
(12) Patent Application Publication
(10) Pub. No.: US 2023/0371100 A1
(43) Pub. Date: Nov. 16, 2023

(54) LOCAL DEVICE NETWORK

(71) Applicant: Oshkosh Corporation, Oshkosh, WI (US)

(72) Inventors: Korry D. Kobel, Oshkosh, WI (US); Fredric L. Yutzy, Oshkosh, WI (US)

(73) Assignee: Oshkosh Corporation, Oshkosh, WI (US)

(21) Appl. No.: 18/195,741

(22) Filed: May 10, 2023

Related U.S. Application Data
(60) Provisional application No. 63/340,688, filed on May 11, 2022.

(51) Int. Cl. H04W 76/15; G01S 11/06

(52) U.S. Cl. CPC H04W 76/15; G01S 11/06; H04W 84/18

(57) ABSTRACT

A local fleet connectivity system includes a plurality of machines. Each machine includes an implement and a prime mover configured to drive the implement and is coupled to a connectivity module configured to wirelessly communicate with connectivity modules of the other machines to form a local network. A first machine of the plurality of machines includes a first connectivity module comprising a first local wireless transceiver. At least one of the connectivity modules is configured to communicate with a computing module via an external network. The system further includes a first device coupled to a local wireless module. The local wireless module is configured to wirelessly communicate with the first local wireless transceiver to connect to the local network. The local wireless module is not configured to directly connect to the external network.

### CROSS-REFERENCE TO RELATED PATENT APPLICATIONS

This Application claims the benefit of and priority to U.S. Provisional Pat. No. 63/340688, filed May 11, 2022, which is hereby incorporated by reference herein in its entirety.

### BACKGROUND

Work equipment such as lifts and telehandlers, as well as smaller devices including generators, power tools, and unpowered implements sometimes require tracking, tasking, monitoring, and servicing at a work site. Managers and operators of work equipment typically rely on discrete systems, applications, and methods to perform these functions for each piece of equipment.

### SUMMARY

One exemplary embodiment relates to a local fleet connectivity system including a plurality of machines. Each machine includes an implement and a prime mover configured to drive the implement and is coupled to a connectivity module configured to wirelessly communicate with connectivity modules of the other machines to form a local network. A first machine of the plurality of machines includes a first connectivity module including a first local wireless transceiver. At least one of the connectivity modules is configured to communicate with a computing module via an external network. The system further includes a first device coupled to a local wireless module. The local wireless module is configured to wirelessly communicate with the first local wireless transceiver to connect to the local network. The local wireless module is not configured to directly connect to the external network.

In some aspects of the local fleet connectivity system, the local wireless module communicates via BLE or RFID. In some aspects, the computing module is configured to determine a distance from the first machine to the first device based on a received signal strength indication of a signal from the local wireless module at the first local wireless transceiver. In some aspects, at least one other machine includes a local wireless transceiver, wherein the computing module is configured to estimate a location of the first device based on received signal strength indications of a signal from the local wireless module at each of the local wireless transceivers. In some aspects, the computing module is configured to receive, from the first connectivity module, an indication that the first local wireless transceiver detects a signal from the local wireless module; and update, in response to receiving the indication, a database to associate the first device with the local fleet connectivity system. In some aspects, the computing module is further configured to determine that the first local wireless transceiver has begun detecting the signal after previously not detecting the signal; and send, in response to the determination, a notification to a user device that the local wireless module has recently been detected. In some aspects of the local fleet connectivity system, the computing module is further configured to receive, from the first connectivity module, a second indication that the first local wireless transceiver no longer detects a signal from first local wireless module; update, in response to receiving the second indication, the database to disassociate the first device with the local fleet connectivity system; and send, in response to receiving the second indication, a notification to a user device that the local wireless module has recently been removed from the local fleet connectivity system.

In some aspects, the local wireless module is communicably coupled to a controller of the first device, the local wireless module configured to receive device information from the controller and to transmit the device information to the first local wireless transceiver. In some aspects, the device information includes one or more of a battery charge level, a fuel level, an activation status, or device malfunction information. In some aspects, the local wireless module is communicably coupled to a controller of the first device, and the controller is configured to receive control instructions from the computing module via the first local wireless transceiver and the local wireless module. In some aspects, the system further includes a connectivity hub, wherein the at least one connectivity module is configured to communicate with an external computer network via wireless communication with the connectivity hub.

Another exemplary embodiment relates to a non-transitory computer-readable storage medium having instructions stored thereon. The instructions, upon execution by a processor, cause the processor to receive a first indication from a first connectivity module of a first machine of a local fleet connectivity system indicating that the first connectivity module detects a signal from a local wireless module of a device. The instructions further cause the processor to update a database to associate the device with the local fleet connectivity system in response to receiving first the indication.

In some aspects, the first indication includes a first signal strength indication indicating the strength of the signal detected by the first connectivity module, and the instructions further cause the processor to determine a distance from the first machine to the device based on the first signal strength indication. In some aspects, the instructions further cause the processor to receive a second indication from a second connectivity module of a second machine of the local fleet connectivity system. The second indication indicates that the second connectivity module detects the signal from the local wireless module. The second indication includes a second signal strength indication indicating the strength of the signal detected by the second connectivity module. The instructions further cause the processor to estimate a location of the device based on the first signal strength indication and the second signal strength indication. In some aspects, estimating the location of the device is further based on a GPS location of the first machine and a GPS location of the second machine.

In some aspects, the instructions further cause the processor to transmit control instructions for the device to the first connectivity module, receipt of the control instructions causing the first connectivity module to transmit the control instructions to the local wireless module. In some aspects, the instructions further cause the processor to receive, from the local wireless module via the first connectivity module, device information relating to the device. In some aspects, the device information includes one or more of a battery charge level, a fuel level, an activation status, or device malfunction information. In some aspects, the instructions further cause the processor to determine that the first connectivity module has begun detecting the signal after previously not detecting the signal. The instructions further cause the processor to transmit a notification to a user device that the device has joined the local fleet connectivity system in response to the determination. In some aspects, the instructions further cause the processor to receive, from the first connectivity module, a second indication that the first connectivity module no longer detects a signal from first local wireless module, update, in response to receiving the second indication, the database to disassociate the device with the local fleet connectivity system, and transmit, in response to receiving the second indication, a notification to a user device that the device has recently been removed from the local fleet connectivity system.

Another exemplary embodiment relates to a method of enabling communication between a device and a cloud computing system via a work machine. The method includes connecting, by a work machine via a short-range wireless communication protocol, to a device and connecting, by the work machine via a second wireless communication protocol, to a cloud computing system. The method further includes receiving, by the work machine, information from the device via the short-range wireless communication protocol and transmitting, by the work machine based on receiving the information, the information to the cloud computing system via the second wireless communication protocol.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE FIGURES

FIG. 1 is a schematic representation of a work machine including a machine control module according to an exemplary embodiment.

FIG. 2 is a schematic representation of a local fleet connectivity system, according to an exemplary embodiment.

FIG. 3 is a schematic representation of a local fleet connectivity system with a central connectivity module, according to an exemplary embodiment.

FIG. 4 is a schematic representation of a work site and equipment staging area with a local fleet connectivity system deployed, according to an exemplary embodiment.

FIG. 5 is a picture representation of a work site with a local fleet connectivity system connecting two pieces of equipment, according to an exemplary embodiment.

FIG. 6 is a picture representation of a piece of equipment with a local fleet connectivity system providing connectivity to a remote user, according to an exemplary embodiment.

FIG. 7 is a schematic representation of a work site with a local fleet connectivity system deployed with connectivity to offsite systems, according to an exemplary embodiment.

FIG. 8 is a picture representation of an apparatus configured with a local fleet connectivity system, according to some embodiments.

FIG. 9 is a picture representation of various graphical user interfaces associated with the local fleet connectivity system of FIG. 2, according to some embodiments.

FIG. 10 is a picture representation of a work machine with machine specific output data connected to the local fleet connectivity system of FIG. 2, according to some embodiments.

FIG. 11 is a picture representation of work machines configured for use in the local fleet connectivity system of FIG. 2, according to some embodiments.

FIG. 12 is a picture representation of a work machine provisioned with an integrated connectivity module and beacon, according to an exemplary embodiment.

FIG. 13 is a combined picture and drawing representation of a work site fleet of work machines and a user device connected to the local fleet connectivity system of FIG. 2, according to an exemplary embodiment.

FIG. 14 is a picture representation of a user interface of the local machine connectivity system of FIG. 2, according to an exemplary embodiment.

FIG. 15 is a flow chart of an exemplary embodiment of a process to deploy the local machine connectivity system of FIG. 2, according to some embodiments.

FIG. 16 is a schematic representation of a work site with a local fleet connectivity system deployed with connectivity to offsite systems, according to an exemplary embodiment.

FIG. 17 is a schematic representation of a work site with a local fleet connectivity system deployed with connectivity to offsite systems, according to an exemplary embodiment.

FIG. 18 is a picture representation of a graphical user interface associated with the local fleet connectivity system of FIG. 16 or FIG. 17, according to some embodiments.

FIG. 19 is a flow chart of an exemplary embodiment of a process for managing the local machine connectivity system of FIG. 16 or FIG. 17, according to some embodiments.

### DETAILED DESCRIPTION

Managers and operators of work equipment typically rely on discrete systems, applications, and methods to perform functions for each piece of equipment. It is therefore desirable to provide a means to electronically connect work equipment on a work site and integrate tracking, tasking, monitoring, and service support functions on a common local fleet connectivity platform to improve efficiency and reduce costs.

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

One exemplary implementation of the present disclosure relates to a local fleet connectivity system (e.g., an interactivity and productivity tool for local fleet connectivity). The local fleet connectivity system may include a network of communicatively connected work machines. Network connections between work machines and other nodes connected to the system may include low energy wireless data networks, mesh networks, satellite communications networks, cellular networks, or wireless data networks. In some implementations, the network of work machines may be a local fleet connectivity system initiated by automatic exchange of networking messages between different machines in the plurality of communicatively connected work machines. In some implementations, a network node is associated with each machine in the plurality of networked machines. In some implementations, a first machine extends a connection to a second machine in proximity to the first machine on a work site to establish a network link at the work site. A work site network may be established among a fleet of work machines at the work site where machines connect with other nearby machines in a mesh network. In some implementations, network access is enabled according to one or more access codes. Access to machine-specific data for one or more machines connected to the network is provided according to the one or more access codes. In some implementations, interconnectivity and productivity related data is exchanged via connectivity modules. The connectivity module may be communicatively connected to a machine controller. The connectivity module may be a self-contained unit. The controller may host one or more interconnectivity and productivity applications. The one or more connectivity and productivity applications hosted by the plurality of controllers may be local instances of a remotely hosted master interconnectivity and productivity application. Connectivity modules may connect to and interconnect through a connectivity hub. In some examples, the connectivity hub may be a device located at a work site that connects to work machines in proximity to the hub via a local network (e.g., a wireless mesh network). In other examples, the connectivity hub may be a remote server.

Referring to the figures generally, various exemplary embodiments disclosed herein relate to systems and methods for a local fleet connectivity system to enhance interactivity and productivity of fleets of work machines on work sites. For example, Bluetooth Low Energy (BLE) Machine-to-Machine (M2M) communication protocols may be used to expand communication at a work site/jobsite via a local fleet connectivity system. In a further example, physical coding sublayer internet protocol (PCS IP) coded instructions (e.g., applications) are used to provide interfaces between work machine software applications in various formats (e.g., MAC, PMA, etc.) and other devices (e.g., mobile user devices). PCS IP may be used, for example, in media independent local fleet connectivity applications within the local fleet connectivity system. In another example, the local fleet connectivity system uses Bluetooth Low Energy (BLE) Machine-to-Machine (M2M) communication protocols at a work site/jobsite to generate and exchange machine driven notifications in a highly efficient and very low error rate by sharing a mesh network. In traditional work site information systems, these notifications are human driven notifications requiring a human operator to physically generate a message and command transmission. As such, traditional work site information systems are inefficient and prone to human error. In another example, machines communicate across a wireless mesh network (e.g., a BLE M2M network) by sending messages across nodes that are created by different machines. One machine may extend a connection with one nearby machine to a network of machines to connect to various machines across a work site. Machines and users may access machine-specific data from those machines that are associated with a common code (e.g. a customer key, identification information, etc.) if accessed using one type of access account (e.g. a customer account with access to all work machines operated by that customer) or access machine-specific data from all of the connected machines if accessed using another type of access account (e.g. a manufacturer account with access to all machines produced by that manufacturer). In a further example, the local fleet connectivity system may provide work site network masking and visibility by means of asset keys to ensure system security and data confidentiality. In another example, the local fleet connectivity system may determine generation and routing of machine generated push messages. These messages may be routed to specific machines based on system-determined or user input criteria.

In the embodiments described, work machines function as micro eco-systems within a macro eco-system. An eco-system may operate at the level of a work site, a collection of work sites supervised by a business unit, a collection of machines operated by a business at multiple sites, a population of machines manufactured by an original equipment manufacturer and operated at many sites by different operators, a business enterprise including many machines from different manufactures supported and monitored by different providers but all interconnected by a common fleet interactivity and productivity platform enabled by interoperable data collection/communications/control/indicator devices provided to each machine in the eco-system. In the embodiments described, the interoperable data collection/communications/control/indicator devices provide near (e.g., at a work site) and far (e.g., remote fleet management node) connectivity and services. Near connectivity and services may include, for example, machine location, machine to machine meshing, service interactions, etc. Far connectivity and services may include, for example, fleet management, incident notification, asset control and status including time and geo-location fencing.

In some implementations, the local fleet connectivity system provides an array of products and functions to improve productivity and reduce ownership costs based on a very high degree of automated machine to machine connectivity that enables exchanges of data and commands and analysis of fleet data that are not possible with traditional work machine tracking, management, telematics systems. For example, the disclosed local fleet connectivity system may create work site ad hoc fleets, automatically check in and check out equipment from a rental or other fleet management application, wirelessly connect with machine components and systems, including machine databuses, to diagnose and troubleshoot faults, remotely determine machine health, functional, and operational status, perform data analytics for user (e.g. users interacting with the system via user devices) and machines connected to the system, and locate individual machines and fleets of machines on any work site at any time.

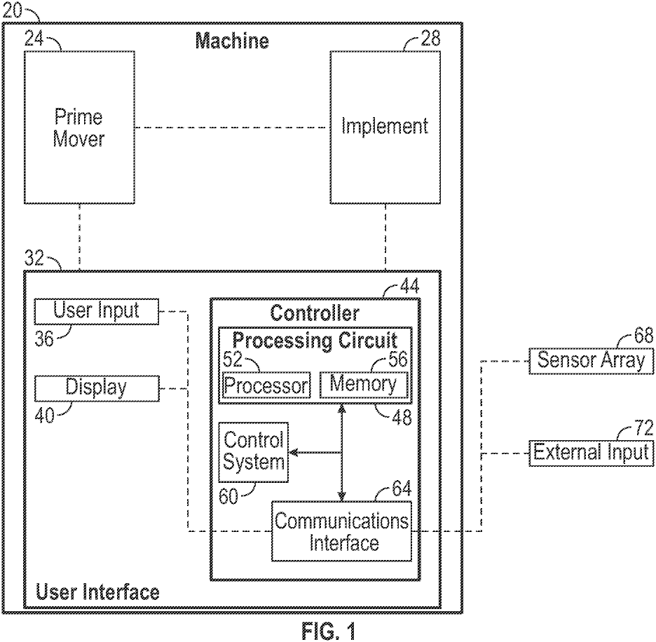

As shown in FIG. 1, a machine, shown as work machine 20 (e.g., a telehandler, a boom lift, a scissor lift, etc.) includes a prime mover 24 (e.g., a spark ignition engine, a compression ignition engine, an electric motor, a generator set, a hybrid system, etc.) structured to supply power to the work machine 20, and an implement 28 driven by prime mover 24. The implement 28 may be any component of the work machine 20 configured to be moved or controlled by the prime mover 24. In some embodiments, the implement 28 is a lift boom, a scissor lift, a telehandler arm, etc.

A user interface 32 is arranged in communication with the prime mover 24 and the implement 28 to control operations of the work machine 20. The user interface 32 includes a user input 36 that allows a machine operator to interact with the user interface 32, a display 40 for communicating to the machine operator (e.g., a display screen, a lamp or light, an audio device, a dial, or another display or output device), and a control module 44.

As the components of FIG. 1 are shown to be embodied in the work machine 20, the controller 44 may be structured as one or more electronic control units (ECU). The controller 44 may be separate from or included with at least one of an implement control unit, an exhaust after-treatment control unit, a powertrain control module, an engine control module, etc. In some embodiments, the control module 44 includes a processing circuit 48 having a processor 52 and a memory device 56, a control system 60, and a communications interface 64. Generally, the control module 44 is structured to receive inputs and generate outputs for or from a sensor array 68 and external inputs or outputs 72 (e.g., a load map, a machine-to-machine communication, a fleet management system, a user interface, a network, etc.) via the communications interface 64.

The control system 60 generates a range of inputs, outputs, and user interfaces. The inputs, outputs, and user interfaces may be related to a jobsite, a status of a piece of equipment, environmental conditions, equipment telematics, an equipment location, task instructions, sensor data, equipment consumables data (e.g. a fuel level, a condition of a battery), status, location, or sensor data from another connected piece of equipment, communications link availability and status, hazard information, positions of objects relative to a piece of equipment, device configuration data, part tracking data, text and graphic messages, weather alerts, equipment operation, maintenance, and service data, equipment beacon commands, tracking data, performance data, cost data, operating and idle time data, remote operation commands, reprogramming and reconfiguration data and commands, self-test commands and data, software as a service data and commands, advertising information, access control commands and data, onboard literature, machine software revision data, fleet management commands and data, logistics data, equipment inspection data including inspection of another piece of equipment using onboard sensors, prioritization of communication link use, predictive maintenance data, tagged consumable data, remote fault detection data, machine synchronization commands and data including cooperative operation of machines, equipment data bus information, operator notification data, work machine twinning displays, commands, and data, etc.

The sensor array 68 can include physical and virtual sensors for determining work machine states, work machine conditions, work machine locations, loads, and location devices. In some embodiments, the sensor array includes a GPS device, a LIDAR location device, inertial navigation, or other sensors structured to determine a position of the equipment 20 relative to locations, maps, other equipment, objects, or other reference points.

In one configuration, the control system 60 is embodied as machine or computer-readable media that is executable by a processor, such as processor 52. As described herein and amongst other uses, the machine-readable media facilitates performance of certain operations to enable reception and transmission of data. For example, the machine-readable media may provide an instruction (e.g., command, etc.) to, e.g., acquire data. In this regard, the machine-readable media may include programmable logic that defines the frequency of acquisition of the data (or, transmission of the data). The computer readable media may include code, which may be written in any programming language including, but not limited to, Java or the like and any conventional procedural programming languages, such as the “C” programming language or similar programming languages. The computer readable program code may be executed on one or more processors, and either local or remote processors. In the latter scenario, the remote processors may be connected to each other through any type of network (e.g., CAN bus, etc.).

In another configuration, the control system 60 is embodied as hardware units, such as electronic control units. As such, the control system 60 may be embodied as one or more circuitry components including, but not limited to, processing circuitry, network interfaces, peripheral devices, input devices, output devices, sensors, etc. In some embodiments, the control system 60 may take the form of one or more analog circuits, electronic circuits (e.g., integrated circuits (IC), discrete circuits, system on a chip (SOCs) circuits, microcontrollers, etc.), telecommunication circuits, hybrid circuits, and any other type of “circuit.” In this regard, the control system 60 may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on). The control system 60 may also include programmable hardware devices such as field programmable gate arrays, programmable array logic, programmable logic devices or the like. The control system 60 may include one or more memory devices for storing instructions that are executable by the processor(s) of the control system 60. The one or more memory devices and processor(s) may have the same definition as provided below with respect to the memory device 56 and processor 52. In some hardware unit configurations, the control system 60 may be geographically dispersed throughout separate locations in the machine. Alternatively, and as shown, the control system 60 may be embodied in or within a single unit/housing, which is shown as the controller 44.

In some embodiments, the control module 44 includes the processing circuit 48 having the processor 52 and the memory device 56. The processing circuit 48 may be structured or configured to execute or implement the instructions, commands, and/or control processes described herein with respect to control system 60. The depicted configuration represents the control system 60 as machine or computer-readable media. However, as mentioned above, this illustration is not meant to be limiting as the present disclosure contemplates other embodiments where the control system 60, or at least one circuit of the control system 60, is configured as a hardware unit. All such combinations and variations are intended to fall within the scope of the present disclosure.

The hardware and data processing components used to implement the various processes, operations, illustrative logics, logical blocks, modules and circuits described in connection with the embodiments disclosed herein (e.g., the processor 52) may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the one or more processors may be shared by multiple circuits (e.g., control system 60 may include or otherwise share the same processor which, in some example embodiments, may execute instructions stored, or otherwise accessed, via different areas of memory). Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. All such variations are intended to fall within the scope of the present disclosure.

The memory device 56 (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory device 56 may be communicably connected to the processor 52 to provide computer code or instructions to the processor 52 for executing at least some of the processes described herein. Moreover, the memory device 56 may be or include tangible, non-transient volatile memory or non-volatile memory. Accordingly, the memory device 56 may include database components, object code components, script components, or any other type of information structure for supporting the various activities and information structures described herein.

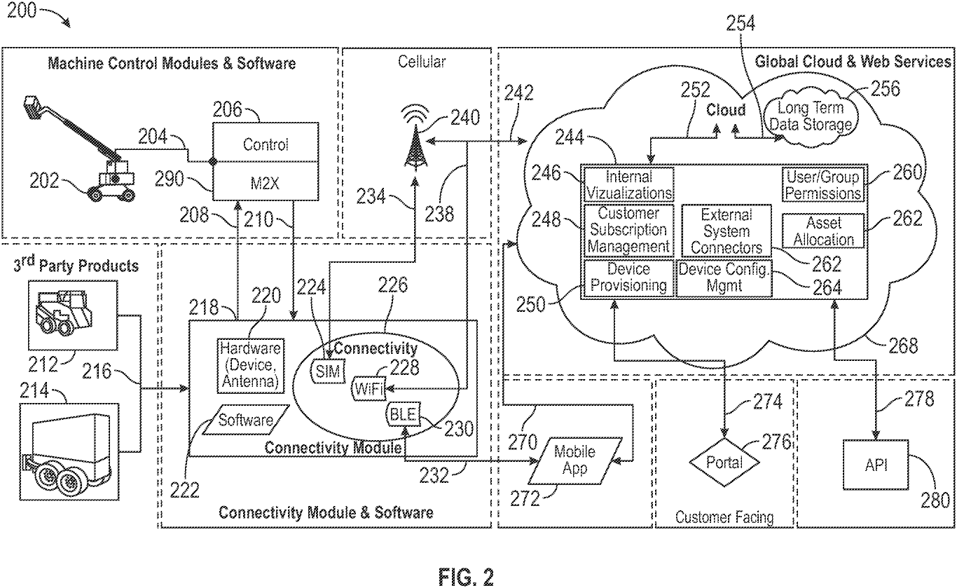

As shown in FIG. 2, a local fleet connectivity system 200 may include one or more work machines 202, each with a control module 206, one or more connectivity modules 218, and one or more network devices hosting, for example, user interfaces 272, network portals 276, application interfaces/application programming interfaces 280, data storage systems 256, cloud and web services, and product development tool and application hubs 244.

The work machine 202 is communicably connected to a control module 206 via a connection 204. Connectivity between the work machine 202 and the control module 206 may be wired or wireless thus providing the flexibility to integrate the control module with the work machine 202 or to temporarily attach the control module 206 to the work machine 202. The control module 202 may be configured or may be reconfigurable in both hardware and software to interface with a variety of work machines 202, 212, 214. The control module 206 may include an integral power source or may draw power from the work machine 202 or another external source of power. Control modules 206 may be installed on or connected to products (e.g., third party products) 212, 214 not configured by the original product manufacturer with a control module 206.

The work machine 202 communicably connects to the local fleet connectivity system 200 via a machine-to-X (M2X) module 290. The M2X module 290 is communicably connected to the control module 206. The M2X module 290 establishes one or more communications channels 208, 210 with a connectivity module 218. The connectivity module 218 provides a plurality of links between one or more work machines 202, 212, 214 and the local fleet connectivity system 200. The local fleet connectivity system applications run by the M2X modules 290 or control modules 206 on one or more work machines 202 to exchange commands, codes (e.g., a customer key) and data between work machines 202, 212, 214, and user devices 272 via the connectivity module 218 to form a network of interconnections among machines, devices, or nodes. Each machine and device connected to the local fleet connectivity system 200 may establish an individual node. Data is exchanged between the different machines and devices by sending the data across the various nodes. For example, a first machine 202 may connect to a second machine 202 that is disposed proximate to the first machine 202. The second machine 202 may be connected to a third machine 202, which may be connected to a fourth machine 202, and so on. Data may be exchanged between any and all of the machines 202 through the various connections via at least one connectivity module 218. Connections between machines and user devices in the local fleet connectivity system 200 may, for example, be provided by a wireless mesh network.

The connectivity module 218 includes hardware 220, further including antennas, switching circuits, filters, amplifiers, mixers, and other signal processing devices for a plurality of wavelengths, frequencies, etc., software hosted on a non-volatile memory component 222, and a communications manager 226. The communications manager 226 includes processing circuits with communications front ends 224, 228, and 230 for one or more signal formats and waveforms including, for example, Bluetooth, Bluetooth low energy, Wi-Fi, cellular, optical, and satellite communications. The connectivity module 218 may function as a gateway device connecting work machine 202 to other work machines 202, 212, 214, other network devices, remote networks 244, 272, 276, and 280, beacons, scheduling or other fleet management and coordination systems.

In some embodiments, the control module 44 of a machine is configured to automatically establish a link (e.g., communicably connect) between various machines 202 and other devices (e.g., user device 272) to each other via at least one connectivity module 218. For example, a control module 44 associated with a machine 202 may be configured to detect other machines, devices, and systems that are capable of communicably connecting to the machine 202 via the connectivity module 218. For example, a first machine 202 may be disposed at a location. A sensor from the sensor array 68 of the first machine 202 may be able to detect at least one other machine 202, 212, 214 disposed at the location (e.g., intercept or sense a signal from other machines indicating their proximity, etc.). In some embodiments, the sensor may detect a plurality of other machines 202, 212, 214. In some embodiments, the first machine may be programmed to connect with any machine it detects. In other embodiments, the first machine may be programmed to only connect with machines of a certain classification. A classification may be any identifiable characteristic of a machine. For example, the classification may be a type of machine (e.g., boom lift, scissor lift, etc.), a phase of a project for which the machine is being used for (e.g., Phase I, Phase II, etc.), a load capacity of the machine (e.g., machines with a load capacity under a predetermined threshold, etc.), a manufacturer of the machine, an operator of a machine, a classification code provided to the machine, a location of the machine, etc. The control module 44 of the first machine 202 may identify the classifications of the detected machines by receiving data indicative of the classification from each of the detected machines via a connectivity module 218. The control module 44 may determine which of the classifications of the detected machines match the classification of the first machine 202 by comparing the classifications of the detected machines with the classification of the first machine 202. Responsive to determining which of the detected machines have matching classifications, the control module 44 may link the first machine 202 with those detected machines. For example, the first machine 202 may be used for Phase II of a project, and the first machine 202 may be programmed to only link with other machines being used for the same phase. Therefore, the control module 44 may be configured to connect the first machine 202 with other detected machines that are also being used for Phase II of the project. In another example, the first machine 202 may be disposed on work site A and may be programmed to only link with other machines disposed on work site A. Therefore, based on the geographic boundaries of work site A and the locations of the detected machines, the control module may be configured to connect the first machine 202 with those detected machines disposed within work site A.

The local fleet connectivity system 200 may communicably connect a plurality of work machines with each other such that data, signals, commands, etc. can be exchanged amongst the machines. The connections between the machines may be established via a mesh network. The mesh network may persist regardless of machines, and other devices, arriving at and leaving from a work site. For example, a local fleet connectivity system 200 may comprise a mesh network connecting a plurality of work machines together that are disposed at a work site. The connectivity between the machines persists even when one of the plurality of work machines leaves the work site or a new work machine comes to the work site. The mesh connecting the plurality of machines may be persistent and constant. The mesh may also be continuously changing to accommodate additional machines or devices or to remove certain machines or devices. In some embodiments, the mesh may remain active such that data may be exchanged at any moment between the plurality of machines. In other embodiments, the mesh may be programmed to only provide connections between the machines at certain times (e.g., during working hours, etc.) or only between certain machines. The mesh may remain active when connecting only work machines. In other embodiments, the mesh may include other devices (e.g., user devices, etc.) between which data may be exchanged.

The local fleet connectivity system 200 provides connectivity between work machines 202, 212, 214 and remotely hosted user interfaces 272, network portals 276, application interfaces/application programming interfaces 280, data storage systems 256, cloud and web services 268, and product development tool and application hubs 244 that function as an Internet of Things (IoT) system for operation, control, and support of work machines 202, 212, 214 and users of work machines. For example, a plurality of work machines 202, 212, 214 disposed at a location that are connected to each other may be configured to connect to at least one user device by the control module 44 via the connectivity module 218. The user device may be disposed at the location or may be disposed at a remote location. Any connections between the machines, the user device, or other network devices, including connections 232, 234, 238, 242, 252, 254, 270, 274, and 278 between nodes connected to the local fleet connectivity system 200, may include, for example, cellular networks, or other existing or new means of digital connectivity. The links between the machines and devices enables data to be exchanged between the plurality of machines 202, 212, 214 and the user device. The local fleet connectivity system 200 allows for the coordination of multiple machines 202, 212, 214 within the same work site, or fleet wide control. For example, a work machine 202 may remotely report the results of a self-inspection to a user via a user device 272.

Product development tool and application hubs 244 may include tools and applications for internal visualizations 246, customer subscription management 248, device provisioning 250, external systems connectors 262, device configuration management 264, user/group permissions 260, asset allocation 258, fleet management, compliance, etc.

According to an exemplary embodiment, within the local fleet connectivity system 200, the control module 44 is configured to receive, via the connectivity module 218, a command from another network device (e.g., a user device). For example, a user of the user device may want a machine 202 to move from a first position to a second position (e.g., move from a first location to a second location, move from an inactive/storage position to an active/operational position, etc.). The control module 44 may receive a command indicating the task of moving from the first position to the second position from the user device via the connectivity module 218. Responsive to receiving the command, the control module 44 may activate the machine to perform the task.

In another embodiment, the control module 44 may be configured to determine that the machine 202 is not capable of performing the task indicated by the command. For example, the control module 44 may be configured to determine a battery level is too low, a part of the machine is broken or missing, the machine is not equipped to perform the task (e.g., the boom of the boom lift is not long enough, the load of the task exceeds the load capacity of the machine, etc.), etc. For example, to detect a low batter level, the control module 44 may be configured to receive a low voltage or no voltage indicating that the machine has no, or too little, power. To detect a load exceeds the load capacity of the machine, the control module 44 may be configured to receive an indication from a sensor (e.g., a pressure sensor) that the pressure applied to the machine 202 is above the predetermine load capacity. Other sensors on the machine 202 may indicate when a part is broken or missing.

Responsive to determining the machine 202 is not capable of performing the task indicated by the command, the control module 44 may be configured to generate a notification indicating the machine is not capable of performing the task. The notification may include details regarding the specific machine (e.g., machine number, time spent at the location, specific location of machine at the location, load capacity, etc.). The notification may include details regarding the task indicated by the command. The notification may include details regarding why the machine is not capable of performing the task (e.g., broken parts, wrong machine, low battery, etc.). If the machine malfunctioned (broken part, low battery, parts aren’t moving properly, etc.), the notification may include instructions on how to fix the problem, which part needs repair, where to buy a replacement part, etc. The control module 44 may be configured to transmit the notification to a user device, or other network device, to notify a user of the inability to perform the task.

In some embodiments, the control module 44, via the connectivity module 218, may be configured to identify a different machine that is capable of performing the task indicated by the command. For example, the control module 44 may be configured to receive data from a second machine 202 indicating all parts are functioning properly (e.g., data from a self-inspection from the second machine 202), the battery is fully charged, the load capacity exceeds the load of the task, etc. The control module 44 may be configured to recommend the second machine 202 as a replacement for the first machine 202 to the user device. In another embodiment, the control module 44 may be able to automatically send, via the connectivity module 218, the command to the second machine 202.

In another embodiment, when the control module 44 determines a machine 202 is malfunctioning, the control module 44 may be configured to designate the machine 202 as inoperable. Based on the designation, the control module 44 may be configured to actuate a visual indicator (e.g., a light, a beacon, etc.). The visual indicator may be indicative of an inoperable state. In some embodiments, a specific visual indicator may correspond to a specific malfunction. For example, the control module 44 may be configured to change a color of a light, change a pulse of the light, change the number of lights, etc. based on what caused the malfunction. For example, a steady red light may indicate a low battery and a flashing red light may indicate a broken part.

In some embodiments, when a machine 202 is designated as inoperable, the machine 202 may be removed from the location. The control module 202 may be configured to determine that the machine 202 is no longer at the location. For example, the control module 44 may have a GPS system that can determine when the machine 202 is no longer at the site. Upon removal, the control module 44 may be configured to disconnect the machine from the other machines at disposed at the location.

According to another exemplary embodiment, within the local fleet connectivity system 200, the control module 44 is configured to receive, via the connectivity module 218, a request from a network device (e.g., a user device) to access machine-specific data corresponding to a plurality of linked machines. In some embodiments, the machine-specific data provided to the network device responsive to receiving the request is limited based on the machine or based on the type of data. For example, a user may have access to only a subset of the plurality of machines. The control module 44 may be configured to identify at least one of the plurality of machines is associated with the user based on an access indicator included in the request. The access indicator may be any information indicative of an association of the machine with the user. For example, the access indicator may be an access code, a customer key, user credentials (user name and password), identification information, the type of account being used (e.g., customer account, manufacturer account, technician account, etc.), etc. Memory device 56 of the control module 44 may be configured to store instructions regarding which machines are associated with which access indicator. The control module 44 may be configured to compare the access indicator received via the request with the instructions stored in the memory device 56 to determine which machine-specific data to provide to the user device. Upon identification of which machines are associated with the access indicator, the control module 44, via the connectivity module 218, may be configured to provide machine-specific data corresponding to the identified machines to the user device.

In another example, a user may have access to all of the plurality of machines, but only to specific information. For example, a customer may only have access to current data (e.g., e.g., current battery level, current location on a job site, current authorized operators, etc.). A manufacturer may have access to all data, including current data and historical data (e.g., average battery life, previous jobs completed, results of previously-performed self-inspections, etc.). Similar to the example above, the control module 44 may be configured to identify a subset of the machine-specific data that is associated with an access indicator that is included in the request and provide that subset of machine-specific data to the user device.

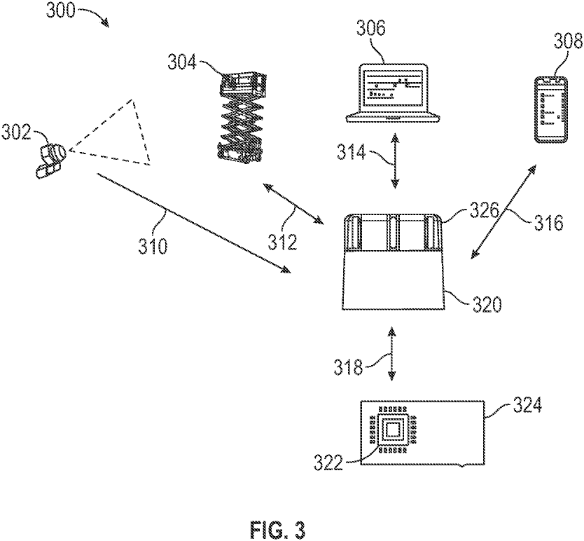

FIG. 3 shows a local fleet connectivity system 300, according to an exemplary embodiment. As shown in FIG. 3, the connectivity module 320 functions as a communications interface between the control system 322 of the work machine 324 and other elements connected to the local fleet connectivity system 200. The connectivity module 320 may be part of the work machine 324 or may be physically coupled with the work machine 324. In some embodiments, the connectivity module 320 includes a beacon, shown as light 326. The connectivity module 320 may exchange commands and data 318 with the control system 322, sensor data 310 with auxiliary sensors 302, machine data 312 with another machine 304, commands and data 314 with a node or portal 306, and commands and data 316 with a user device 308 running an application for the local fleet connectivity system 300.

Any of the data 310, 312, 314, 316, 318 exchanged between the various connected devices and the connectivity module 320 may be further exchanged with other connected devices. For example, sensor data 310 from the auxiliary sensors 302 may be received by the connectivity module 320 and then further transmitted to the user device 308 such that a user of the user device 308 can see what the auxiliary sensor 302 detected. In response to viewing the data via the user device 308, the user can provide a command via the user device 308 that can be received by the connectivity module 320 and further transmitted to the device being commanded. For example, a sensor 302 may detect that the battery of the work machine 304 is getting low. The sensor 302 may send the low battery reading to the connectivity module 320, which is further transmitted to the user device 308. Upon receiving the indication of the lower battery, the user may command the work machine 304 to return to its storing orientation (e.g., collapsed state). The command may be sent to the work machine 304 via the connectivity module 320. Any of the devices 302, 304, 306, 308, 324 may communicate with each other via the communication module 320.

The local fleet connectivity system 200 allows for the coordination of multiple machines 324, 304 within the same work site, or a fleet wide control. For example, if a first work machine 304 is required to accomplish a task collaboratively with a second work machine 324, a user interacting with a user device 308 may provide commands to the first work machine 304 and second work machine 324 to execute the task in collaboration.

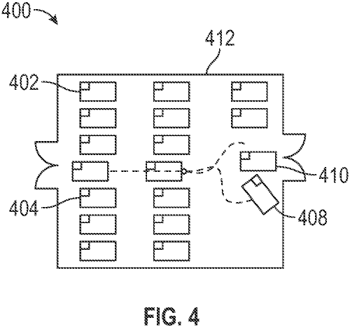

Referring now to FIG. 4, a fleet connectivity system 400 is shown, according to an exemplary embodiment. As discussed above, the fleet connectivity system 400 may be deployed at a work site 412 to control a fleet of work machines 402, 404, 408, 410, so as to collaboratively perform tasks requiring more than one work machine 408, 410. For example, a user may wish to move the work machine 410 from its stored position on the left of the work site 412 out the door on the right of the work site. Components of the fleet connectivity system 400 (e.g., a network access point, a system access point, a connectivity hub, work machines having a connectivity module, etc.) may communicate with both the work machine 408 and the work machine 410, causing the work machine 408 to move out of the way of the work machine 410, so that the work machine 410 can move past the work machine 408 and out the doorway.

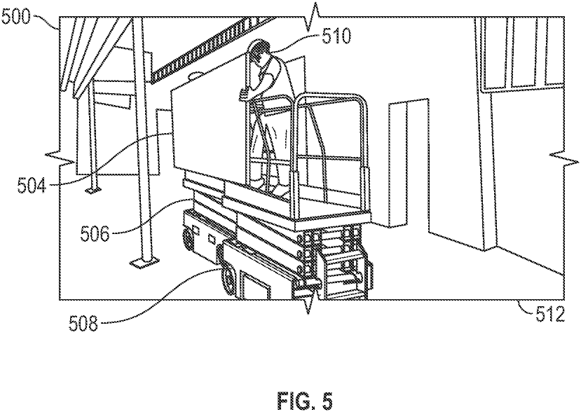

Referring now to FIG. 5, a fleet connectivity system 500 is shown, according to an exemplary embodiment. As discussed above, the fleet connectivity system 500 may be communicably coupled to a plurality of work machines 506, 508 (e.g., via a plurality of connectivity modules), such that the work machines 506, 508 may collaboratively perform tasks on a jobsite 512. For example, as shown in FIG. 5 the fleet connectivity system 500 may be used to replace a section of drywall 504 that is too large to be handled by a single work machine 508. Components of the fleet connectivity system 500 (e.g., a network access point, a system access point, a connectivity hub, etc.) may communicate with both the work machine 506 and the work machine 508 and cause them to move at the same speed and in the same direction so that a user 510 on each work machine 506, 508 may hold the drywall 504 while the work machines 506, 508 are moving. In this regard, communication between components of the fleet connectivity system and the work machines 506, 508 may prevent the work machines 506, 508 from being separated so that the users 510 do not drop the drywall 504.

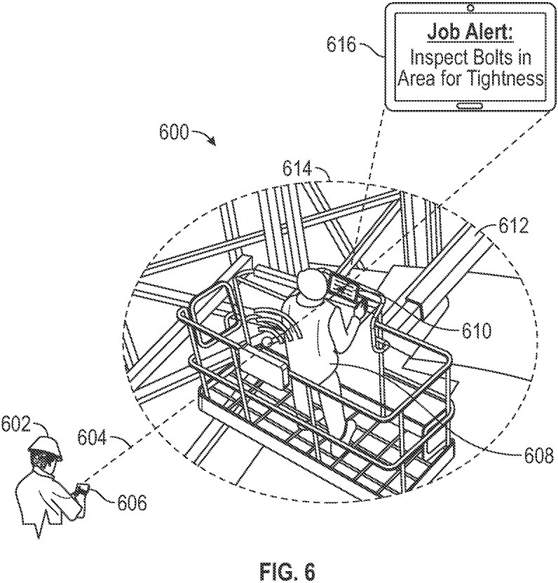

As shown in FIG. 6, a remote user 602 of a local fleet connectivity system 600 can send messages and data 604 from a remote device 606 to an onsite user 608 on a jobsite 614. The messages and data 604 may be received by the control system 610 of a work machine 612 via a connectivity module and displayed via a user interface on an onboard display 616. The remote user 608 may send work instructions to the onsite user 608, informing the onsite user 608 of talks to be performed using the work machine 612. For example, as shown in FIG. 6, the remote user 602 may send instructions to the onsite user 608 to use the work machine 612 to inspect bolt tightness in the area. The instructions may be displayed for the onsite user 608 on the onboard display 616. This allows the onsite user 608 to receive and view the instructions without the need to call the remote user 602 or write the instructions down. Because the work machine 612 is connected to the remote device 606 (e.g., via a connectivity module 218) the remote user 602 may receive the location of the work machine 612, as well as other work machines on the jobsite 614, and may use the location information to determine the instructions to send.

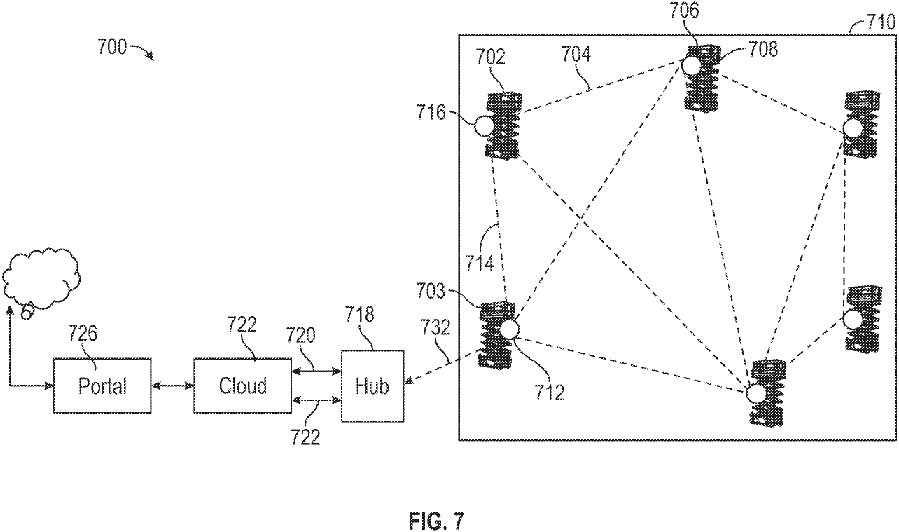

As shown in FIG. 7, a local fleet connectivity system 700 includes a connectivity hub 718, according to an exemplary embodiment. In some embodiments, the connectivity hub 718 includes a connectivity module 218. In some embodiments, the connectivity hub 718 is configured to communicably connect with one or more connectivity module equipped machines 702, 703 706 in proximity to the connectivity hub 718. In some embodiments, the connectivity hub 718 is configured to broadcast a work site identification signal. In some embodiments, the connectivity hub 718 is configured to connect work site machines 702, 706 connected to the local fleet network to an external internet feed 720. In some configurations, the connectivity hub 718 is configured as a gateway to one or more communications systems or network systems to enable exchanges of data between connectivity modules 708, 712, 716 of machines on the work site 710 connected to the local fleet connectivity network 704, 714, 732 and nodes 722, 726 external to the work site.

In some embodiments, connectivity hub 718 has a connectivity module 218 to (a) provide the functionalities described herein in place of or in addition to a machine that has a connectivity module 218, (b) broadcast a site identifier, or (c) connect to an external internet to flow data to and from the jobsite that is provided across the mesh.

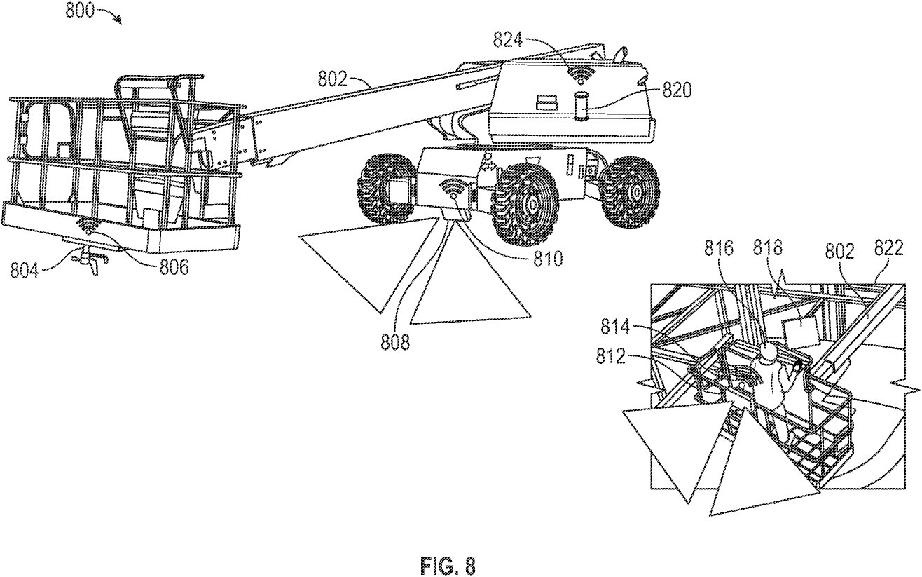

Referring to FIG. 8, a local fleet connectivity system 800 is shown, according to an exemplary embodiment. Sensors 804, 808, 812, 820 may be coupled to a work machine 802 on a jobsite 822. The sensors 804, 808, 812, 820 may be, for example, object detection sensors, environmental sensors (e.g., wind speed, temperature sensors), and tagged consumable sensors. The sensors 804, 808, 812, 820 may be connected to and may send data via the local fleet connectivity system 800 via wireless connections 806, 810, 814, 824. The sensor data may be displayed or may be used to generate messages for display on an onboard display 818 for a user 816 of the work machine 802. The onboard display 818 may receive the sensor data via a direct wired or wireless connection to the sensors. Alternatively, the sensors may communicate with the onboard display through the local fleet connectivity system 800 (e.g., via a connectivity module 218). Sensor data from various work machines may be combined to map the jobsite 822 and to determine if environmental conditions are safe for using the work machines. Sensor data from the tagged consumable sensors 820 may be used to determine, for example, when tagged consumables must be replaced.

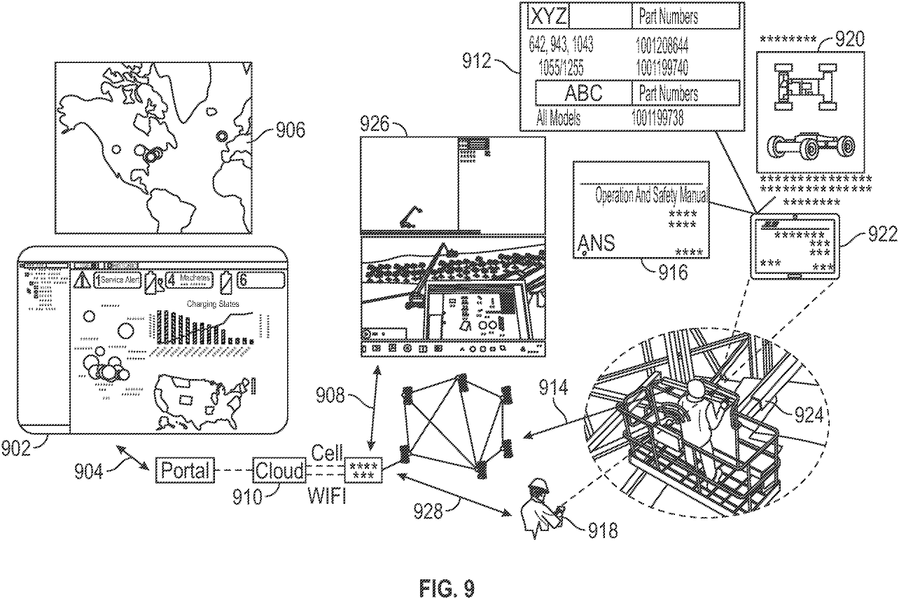

As shown in FIG. 9, various user interfaces are available to be displayed on a remote user device 918 and an onboard display 922 of a work machine 924. A connectivity hub 910 may send and receive data 928, 908, 904, 914 including the user interfaces 902, 906, 912, 916, 926, 920. The user interface 906 is a heat map of locations of a plurality of work machines. The user interface 902 is a machine status display that shows the battery level, location, and alerts relating to a plurality of work machines. User interface 926 shows a digital twin of a work machine that updates based on sensor data of an associated work machine. User interface 912 is a list of part numbers for the work machine 924. User interface 916 is an operation and safety manual for the work machine 924. User interface 920 is a detailed schematic of the work machine 924.

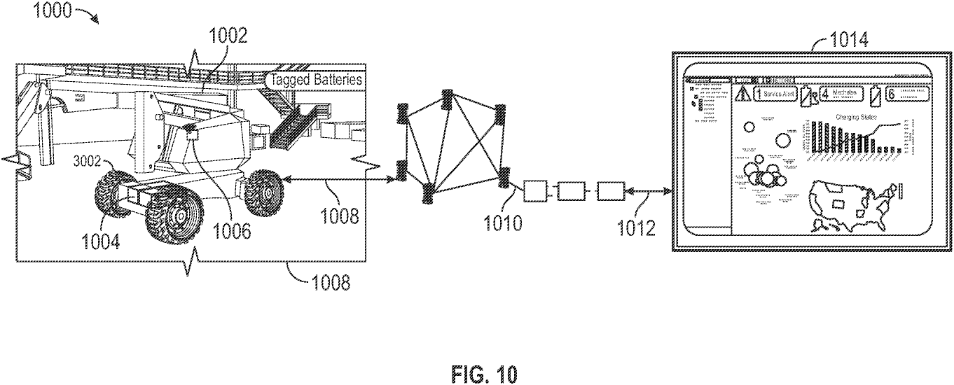

As shown in FIG. 10, a tagged consumable tracking system 1000 is shown. A work machine 1002 on a jobsite 1008 includes tagged consumables 1004 (e.g., batteries connected to battery charger 1006). The machine 1002 sends and receives data 1009 to and from the connectivity hub 1010. The connectivity hub 1010 sends and receives data 1012 to and from a remote device and produces a user interface 1014. Data regarding the tagged consumables 1004 may be communicated via the user interface 1014 via the connectivity hub 1010. For example, battery charge state and battery health may be displayed via the user interface 1014. When the battery health falls below a predetermined state, for example, when the battery is only able to hold half of its original charge, the connectivity hub 1010 may send an alert via the user interface 1014 indicating that the battery should be replaced.

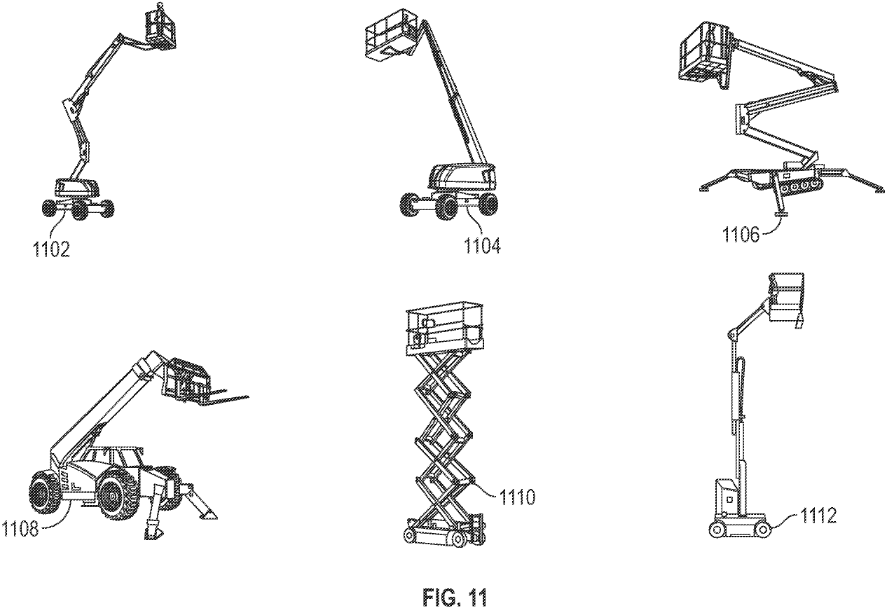

As shown in FIG. 11, the local fleet connectivity systems and methods described above may be implemented using various work machines 20 such as an articulating boom lift 1102 as shown in FIG. 11, a telescoping boom lift 1104 as shown in FIG. 11, a compact crawler boom lift 1106 as shown in FIG. 11, a telehandler 1108 as shown in FIG. 11, a scissor lift 506, 508, and 1110 as shown in FIGS. 5 and 11, and/or a toucan mast boom lift 1112 as shown in FIG. 11.

According to the exemplary embodiment shown in FIG. 11, the work machines 20 (e.g., a lift device, articulating boom lift 1102, telescoping boom lift 1104, compact crawler boom lift 1106, telehandler 1108, scissor lift 1110, toucan mast boom lift 1112) include a chassis (e.g., a lift base), which supports a rotatable structure (e.g., a turntable, etc.) and a boom assembly (e.g., boom). According to an exemplary embodiment, the turntable is rotatable relative to the lift base. According to an exemplary embodiment, the turntable includes a counterweight positioned at a rear of the turntable. In other embodiments, the counterweight is otherwise positioned and/or at least a portion of the weight thereof is otherwise distributed throughout the work machines 20 (e.g., on the lift base, on a portion of the boom, etc.). As shown in FIG. 11, a first end (e.g., front end) of the lift base is supported by a first plurality of tractive elements (e.g., wheels, etc.), and an opposing second end (e.g., rear end) of the lift base is supported by a second plurality of tractive elements (e.g., wheels). According to the exemplary embodiment shown in FIG. 11, the front tractive elements and the rear tractive elements include wheels; however, in other embodiments the tractive elements include a track element.

As shown in FIG. 11, the boom includes a first boom section (e.g., lower boom, etc.) and a second boom section (e.g., upper boom, etc.). In other embodiments, the boom includes a different number and/or arrangement of boom sections (e.g., one, three, etc.). According to an exemplary embodiment, the boom is an articulating boom assembly. In one embodiment, the upper boom is shorter in length than lower boom. In other embodiments, the upper boom is longer in length than the lower boom. According to another exemplary embodiment, the boom is a telescopic, articulating boom assembly. By way of example, the upper boom and/or the lower boom may include a plurality of telescoping boom sections that are configured to extend and retract along a longitudinal centerline thereof to selectively increase and decrease a length of the boom.

As shown in FIG. 11, the lower boom has a first end (e.g., base end, etc.) and an opposing second end (e.g., intermediate end). According to an exemplary embodiment, the base end of the lower boom is pivotally coupled (e.g., pinned, etc.) to the turntable at a joint (e.g., lower boom pivot, etc.). As shown in FIG. 11, the boom includes a first actuator (e.g., pneumatic cylinder, electric actuator, hydraulic cylinder, etc.), which has a first end coupled to the turntable and an opposing second end coupled to the lower boom. According to an exemplary embodiment, the first actuator is positioned to raise and lower the lower boom relative to the turntable about the lower boom pivot.

As shown in FIG. 11, the upper boom has a first end (e.g., intermediate end, etc.), and an opposing second end (e.g., implement end, etc.). According to an exemplary embodiment, the intermediate end of the upper boom is pivotally coupled (e.g., pinned, etc.) to the intermediate end of the lower boom at a joint (e.g., upper boom pivot, etc.). As shown in FIG. 11, the boom includes an implement (e.g., platform assembly) coupled to the implement end of the upper boom with an extension arm (e.g., jib arm, etc.). In some embodiments, the jib arm is configured to facilitate pivoting the platform assembly about a lateral axis (e.g., pivot the platform assembly up and down, etc.). In some embodiments, the jib arm is configured to facilitate pivoting the platform assembly about a vertical axis (e.g., pivot the platform assembly left and right, etc.). In some embodiments, the jib arm is configured to facilitate extending and retracting the platform assembly relative to the implement end of the upper boom. As shown in FIG. 11, the boom includes a second actuator (e.g., pneumatic cylinder, electric actuator, hydraulic cylinder, etc.). According to an exemplary embodiment, the second actuator is positioned to actuate (e.g., lift, rotate, elevate, etc.) the upper boom and the platform assembly relative to the lower boom about the upper boom pivot.

According to an exemplary embodiment, the platform assembly is a structure that is particularly configured to support one or more workers. In some embodiments, the platform assembly includes an accessory or tool configured for use by a worker. Such tools may include pneumatic tools (e.g., impact wrench, airbrush, nail gun, ratchet, etc.), plasma cutters, welders, spotlights, etc. In some embodiments, the platform assembly includes a control panel to control operation of the work machines 20 (e.g., the turntable, the boom, etc.) from the platform assembly. In other embodiments, the platform assembly includes or is replaced with an accessory and/or tool (e.g., forklift forks, etc.).

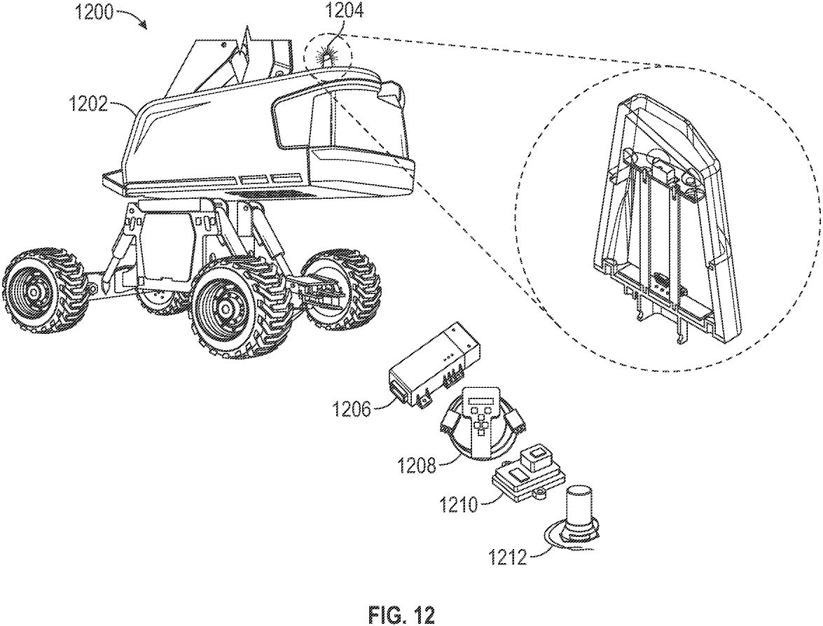

Referring now to FIG. 12, a work machine 1202 may be provisioned with an integrated connectivity module 1204 configured to connect to the local fleet connectivity system 1200. The integrated connectivity module 1204 may be configured to perform the functions of multiple devices that are often installed as separate components in traditionally provisioned work machines 1202. The functions and components provided in the integrated connectivity module 1204 can include telematics 1206, analytics 1208, communications 1210, visual and aural indicators 1212 (e.g., a warning beacon), etc.

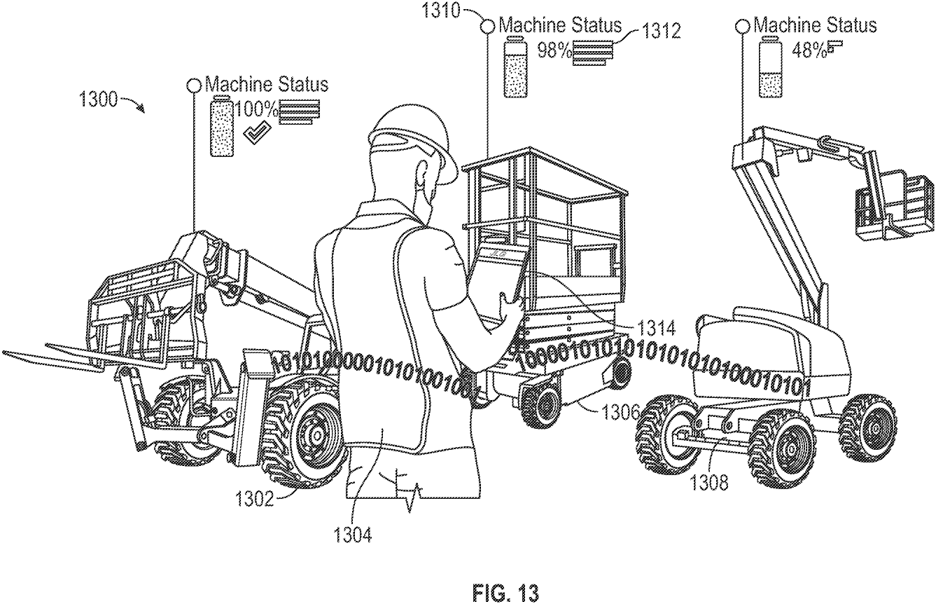

Referring now to FIG. 13, work machines 1302, 1306, 1308 equipped with connectivity modules 218 may form a local fleet connectivity network at a work site with machines 1302, 1306, 1308 and user devices 1314 acting as nodes 1310 on the network. The local fleet connectivity network at a work site connects to the local fleet connectivity system and provides machines 1302, 1306, 1308 and users 1304 access to data shared via the local fleet connectivity system. Available data include, for example, machine statuses 1312 (e.g., battery life, malfunctioning parts, etc.), machine locations, machine availability, etc.

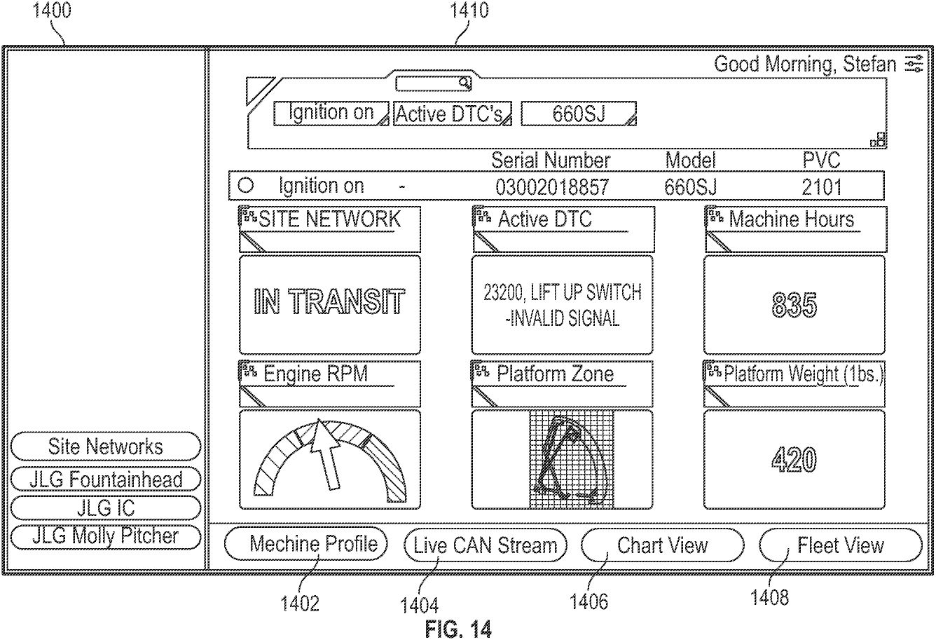

Referring now to FIG. 14, the local fleet connectivity system 200 may generate user interface 1400. The user interface 1400 may be presented to the user as a user view 1410 depending on the role of the user and the nature of a task. The user view may include textual and graphic representations of, for example, a machine profile 1402, a machine databus stream 1404, a machine position, configuration, or state 1406, data related to a fleet of machines 1408, etc.

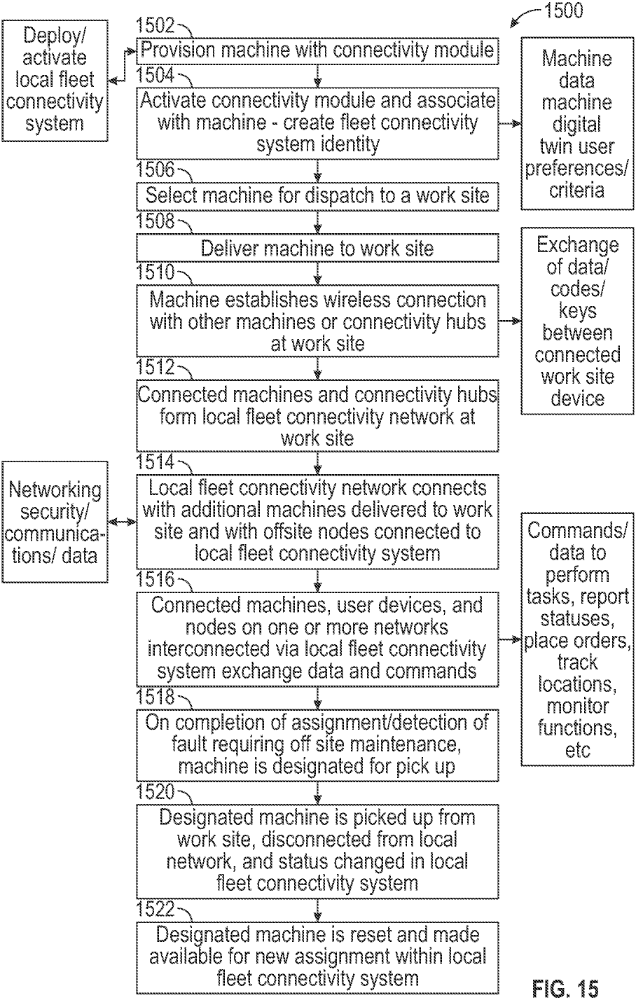

Referring now to FIG. 15, in some embodiments, a method 1500 for providing local fleet connectivity for groups of work machines associated with one or more work sites includes, at operation 1502, providing a machine with a connectivity module. The connectivity module enables the machine to communicably connect with other devices such that data, commands, etc. can be exchanged. For example, a machine with the connectivity module may send data to another device (e.g., a user device) regarding the machine’s battery level, whether any parts of the machine need repair or a replacement, how long the machine has been in use, etc. At operation 1504, the connectivity module is activated and associated with the machine. Activation and association of the connectivity module may provide system level visibility to a digital twin of the machine, machine location, status, and digital records for the machine that are stored onboard the machine or remotely. User access to machine control and machine data may be provided according to access permissions. In some embodiments, only a subset of the data related to the machine is accessible to the user. For example, an operator may only be able to access current operational status of the machine (e.g., current battery level), while the manufacturer may be able to access historical operational statuses of the machine (hours of work performed on a single battery charge). The data accessible by the user may be determined based on an access indictor used to access the data. The access indicator may be any information indicative of an association of the machine with the user. For example, the access indicator may be an access code, a customer key, user credentials (user name and password), identification information, the type of account being used (e.g., customer account, manufacturer account, technician account, etc.), etc. For example, if the account used to access the data is associated to an individual operator, only the current operational status may appear. If the account is associated with the company that manufactured the machine, different information may be accessible. A memory device of control module may store instructions regarding which machines are associated with which access indicator. The control module 44 may be configured to compare the access indicator with the instructions stored in the memory device.

In some embodiments, at operation 1506, the machine is selected for dispatch to a work site. At operation 1508, the machine is delivered to the work site. At operation 1510, the machine links (e.g., communicably connects) with other machines or connectivity hubs at the work site by establishing a wireless connection with the other machines or connectivity hubs. Operation 1510 may include establishing, by at least one control module via at least one connectivity module, a connection between a plurality of machines disposed at a location. The link between a plurality of machines enables an exchange of data, codes, keys, etc. between the connected machines. In some embodiments, establishing the connection may be performed automatically. For example, when the machine arrives at the work site, the control module may detect a plurality of machines disposed at a location. In some embodiments, the machine may connect with all the detected machines. In other embodiments, the machine may only connect with a subset of the detected machines. A control module of a machine may receive input indicating which machines it may connect to. For example, only machines of the same type (e.g., boom lifts) may connect, only machines made by the same manufacturer may connect, or only machines within a predetermined geographical area may connect to each other. For example, a control module of the machine may receive an input identifying a designated geographical area on a map that corresponds to a work site. The input may indicate that any machine disposed within the designated geographical area may be connected with each other. In other words, the input may define the boundaries of the local fleet connectivity system. In another example, the input may indicate that any machine within a predetermined radius may connect with each other.

In other embodiments, the machine may connect with other machines based on an identification or classification of the machine. The classification may be based on a variety of factors including, but not limited to, a phase of a project the machine is being used for (e.g., a phase I machine), type of machine (e.g., boom lift, telehandler, scissor lift, etc.), size of machine (e.g., based on weight, dimensions, load capacity, etc.), who is authorized to use the machine (e.g., all machines operated by person A can be connected), etc. For example, a first subset of machines may be identified as A machines and a second subset of machines may be identified as B machines. The control module may identify a classification of each of the plurality of machines. The control module may then determine which of the plurality of machines have a classification that matches the classification of the delivered machine. Responsive to determining which of the plurality of machines have matching classifications, the control module may link the plurality of machines that have the matching classifications together via at least one connectivity module.

Data, codes, keys, etc. may be shared among the connected machines in order to facilitate the combination and organization of information regarding all of the machines. For example, once connected, a person authorized to access all information regarding the group of machines will have access to the information regarding all of the connected machines (e.g., locations, statuses, repair requirements, etc.) without having to search for each machine individually.

At operation 1512, the connected machines and connectivity hubs form a local fleet connectivity network at the work site. Each of the machines and hubs may comprise a node of the local fleet connectivity network. At operation 1514, the local fleet connectivity network connects with additional machines and network devices (e.g., a user device) delivered to the work site and with offsite nodes connected to the local fleet connectivity system. Connecting to the offsite notes enables the machines to provide data to devices at a remote location. For example, when a machine malfunctions, a notification may be transmitted to a technician at a remote location via a user device. The notification, via an application associated with the connectivity module, may provide the technician with specific details regarding the specific machine and the specific malfunction (e.g., components of the machine that need to be replaced, how to fix the problem, where to buy parts, etc.).

In some embodiments, at operation 1516, connected machines, user devices, and nodes on one or more networks interconnected via the local fleet connectivity system exchange data and commands. The exchange of data and commands may enable the system to perform tasks, report statuses, place orders, track locations, monitor functions, etc. according to system provided permissions. For example, the system may be able to receive inputs and provide outputs based on the type of account that is being used to access the machine. For example, an operator working at a work site may be able to perform different tasks and view different data than a technician or manufacturer of the machine. In some embodiments, a machine accessed by the operator may be able to track locations of all the connected machines at the specific work site, while a machine accessed by the manufacturer may be able to track locations of all connected machines at any work site. For another example, a machine accessed by the operator may be able to transmit a signal indicating a malfunction in the machine. The machine accessed by a technician may be able to access details regarding the malfunction, troubleshoot the malfunction, access instructions on how to best fix the malfunction, and assist in ordering parts required to repair the machine based on the malfunction.

In some embodiments, at operation 1516, a control module of a machine may receive, via a connectivity module, a command from at last one user device the command may include a task to be performed. Based on the command, the control module may activate the machine to perform the task. For example, the task may be for the machine to move from a first location at a work site to a second location at a work site. The control module may activate an engine of the machine such that the machine moves to the identified location. In some embodiments, instead of completing a task, the control module may determine the machine is not capable of performing the task. For example, the control module 44 may determine a battery level is too low, a part of the machine is broken or missing, the machine is not equipped to perform the task (e.g., the boom of the boom lift is not long enough, the load of the task exceeds the load capacity of the machine, etc.), etc. For example, to detect a low batter level, the control module 44 may receive a low voltage or no voltage indicating that the machine has no, or too little, power. To detect a load exceeds the load capacity of the machine, the control module 44 may receive an indication from a sensor (e.g., a pressure sensor) that the pressure applied to the machine 202 is above the predetermine load capacity. Other sensors on the machine 202 may indicate when a part is broken or missing.

Responsive to determining the machine 202 is not capable of performing the task indicated by the command, the control module 44 may generate a notification indicating the machine is not capable of performing the task. The notification may include details regarding the specific machine (e.g., machine number, time spent at the location, specific location of machine at the location, load capacity, etc.). The notification may include details regarding the task indicated by the command. The notification may include details regarding why the machine is not capable of performing the task (e.g., broken parts, wrong machine, low battery, etc.). If the machine malfunctioned (broken part, low battery, parts aren’t moving properly, etc.), the notification may include instructions on how to fix the problem, which part needs repair, where to buy a replacement part, etc. The control module 44 may transmit the notification to a user device, or other network device, to notify a user of the inability to perform the task.

In some embodiments, the control module 44, via the connectivity module 218, may identify a different machine that is capable of performing the task indicated by the command. For example, the control module 44 may receive data from a second machine 202 indicating all parts are functioning properly (e.g., data from a self-inspection from the second machine 202), the battery is fully charged, the load capacity exceeds the load of the task, etc. The control module 44 may recommend the second machine 202 as a replacement for the first machine 202 to the user device. In another embodiment, the control module 44 may automatically send, via the connectivity module 218, the command to the second machine 202.

In another embodiment, when the control module 44 determines a machine 202 is malfunctioning, the control module 44 may designate the machine 202 as inoperable. Based on the designation, the control module 44 may actuate a visual indicator (e.g., a light, a beacon, etc.). The visual indicator may be indicative of an inoperable state. In some embodiments, a specific visual indicator may correspond to a specific malfunction. For example, the control module 44 may change a color of a light, change a pulse of the light, change the number of lights, etc. based on what caused the malfunction. For example, a steady red light may indicate a low battery and a flashing red light may indicate a broken part.

In some embodiments, at operation 1518, at the completion of an assignment or at the detection of a fault condition requiring off site maintenance, the machine is designated for pick up. In some embodiments, the machine may send a notification to a remote user device indicating that the machine is to be removed from the work site. At operation 1520, the designated machine is picked up at the work site. Upon pick up, the machine may be disconnected from the local network. The disconnection may be done manually (e.g., user turns off or disengages the connectivity module of the machine) or automatically (e.g., machine determines it is no longer on the work site and disconnects from the network). For example, the control module 44 may have a GPS system that can determine when the machine 202 is no longer at the site. Upon removal, the control module 44 may be configured to disconnect the machine from the other machines at disposed at the location. In some embodiments, a new status of the machine is identified in the local fleet connectivity system. For example, when connected to the local fleet connectivity system, the machine may indicate a status of connected, operational, ready, etc. Upon disconnection, the machine may indicate a status of disconnected, inoperable, being serviced, etc.

In some embodiments, at operation 1522, the designated machine is reset (e.g., fueled, charged, serviced, repaired, upgraded, etc.) and made available for a new assignment within the local fleet connectivity system. In some embodiments, the machine may be returned to the same work site and connected to the same local fleet connectivity system. In other embodiments, the machine may be sent to a new work site and connected to a new local fleet connectivity system. Method 1500 may be performed any number of times for any machine and can include any number of local fleet connectivity systems.

In some embodiments, additional devices that do not include connectivity modules 708, 712, 716, including unpowered devices, may be connected to a local network of machines in a local fleet connectivity system via local wireless technology. For example, smaller power tools, unpowered hand tools, and other unpowered implements may be equipped with one or more local wireless modules configured to communicate with local wireless transceivers of the machine connectivity modules 708, 712, 716 as discussed above. The modules may communicate, for example, via Radio-frequency identification (RFID), Bluetooth Low Energy (BLE) or other short-range, low-bandwidth communication protocols. The local wireless modules may not be configured to communicate directly with external computer networks. Instead, the connectivity modules 708, 712, 716 of the machines may act as access points for the local wireless modules to communicate with an external network. In some embodiments, the local wireless modules may be simple RFID or BLE tags coupled to the devices that generate a signal that can be detected by the local wireless transceivers.

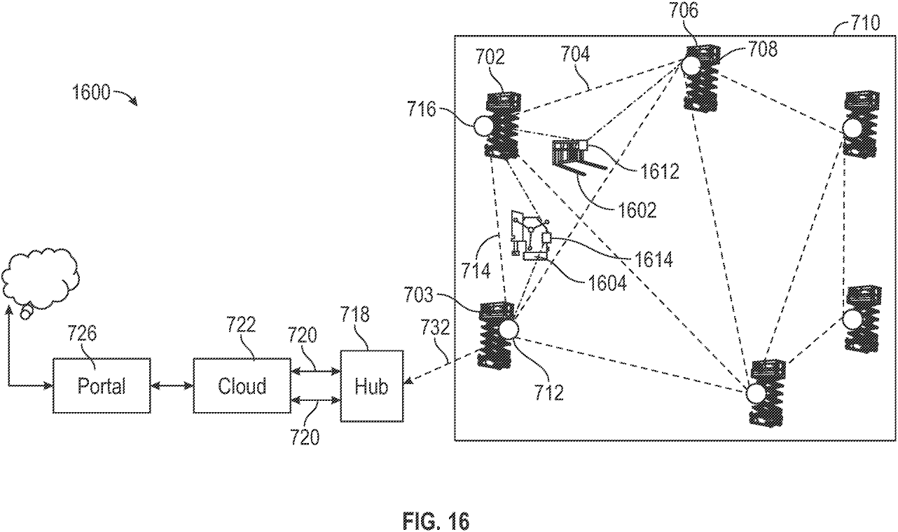

Referring now to FIG. 16, a local fleet connectivity system 1600 is shown, according to some embodiments. The local fleet connectivity system 1600 may be substantially similar to the local fleet connectivity system 700, with the addition of the additional devices 1602 and 1604, respectively shown as a telehandler carriage and a magnetic drill. The additional devices 1602, 1604 each include a local wireless module 1612, 1614 that is configured to wirelessly communicate with a local wireless transceiver of a respective connectivity module 716, 712, 708 of a machine 702, 703, 706. These local wireless modules 1612, 1614, may be low-energy ID tags that may transmit small amounts of information to the local wireless transceivers. For example, the tags may simply transmit to the wireless transceiver the identity of the tag or the identity of the device to which the tag is coupled. Thus, the wireless transceivers in the connectivity modules 708, 712, 716 may detect the presence of the devices 1602, 1604 within a signal radius of the local wireless module 1612, 1614. A BLE tag may have a maximum nominal signal range of about 100 meters. Therefore, a local wireless transceiver detecting a signal from a BLE tag coupled to a device may signify that the device is within about 100 meters of the local wireless transceiver. Similarly, a passive RFID tag may have a maximum signal range of about 10 meters, and a local wireless transceiver detecting the passive RFID tag may signify that the device is within 10 meters of the local wireless transceiver. These simple tags may be more useful for unpowered tools and implements, such as telehandler implements like fork carriages and material buckets, as well as powered implements that do not include controllers configured to communicate with the local wireless modules 1612, 1614, such as powered hand tools like drills and saws. However, these tags can be easily coupled to any device.

As described above, the connectivity modules 708, 712, 716 may be configured to communicate with each other and with a cloud computing system 722 or other external computer network (e.g., the internet), either directly (e.g., through a cellular or satellite connection) or via a connectivity hub 718. The local wireless modules 1612, 1614 may not be configured to communicate or capable of communicating directly with an external computer network and in some embodiments may not be configured to communicate directly with the connectivity hub 718. The local wireless modules 1612, 1614 may, however, communicate with an external computer network through the connectivity modules 708, 712, 716 of the machines 702, 703, 706. Thus, the connectivity modules 708, 712, 716 may act as gateways for the local wireless modules 1612, 1614 to connect to external networks. The local fleet connectivity system may include one or more computing modules (e.g., computers, servers, processing circuits, etc.) communicably connected to the local network via at least one connectivity module 708, 712, 716 or the connectivity hub 281. The computing module may be, for example, a control module 206 of a machine, a processor located in the connectivity hub 718, or a remote server (e.g., in the cloud 722). The computing module may receive data from the local wireless modules 1612, 1614 via the connectivity modules 708, 712, 716 and in some cases may send data and/or instructions to the local wireless modules 1612, 1614 via the connectivity modules 708, 712, 716.

In some embodiments, the computing module may be configured to determine a distance from their machines 706, 703, 702 to the devices 1602, 1604 based on a signal strength indication (e.g., received signal strength indication (RSSI)) of the signal from the local wireless module 1612, 1614 at the local wireless transceiver the connectivity modules 708, 712, 716. A strong RSSI may indicate that the local wireless module 1612, 1614 is close in distance to the connectivity module 708, 712, 716, while a weaker RSSI may indicate that the local wireless module 1612, 1614 is farther away from the connectivity module 708, 712, 716. The local wireless transceiver of the connectivity module 708 may detect a signal from the local wireless module 1612 comprising a BLE tag indicating a low RSSI. The connectivity module 708 may transmit the RSSI to the computing module, and the computing module may determine that the local wireless module 1612, and therefore the device 1602, is just inside the range of the BLE tag, and may determine or estimate that the device 1602 is about 90-100 meters from the machine 706. If there is a stronger RSSI at the connectivity module 708, the computing module may determine that the device 1602 is closer to the machine 706.

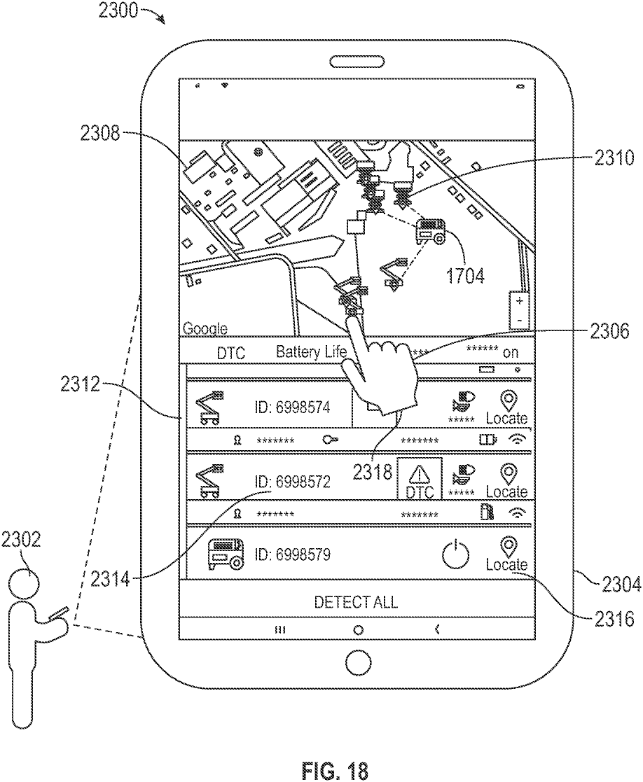

In some embodiments, a signal from a local wireless module 1612, 1614, may be received by more than one connectivity module 708, 712, 716. For example, as shown in FIG. 16, the local wireless module 1612 is connected to connectivity modules 708 and 716 but is not connected and may be out of range of connectivity module 712. Connectivity modules 708 and 716 may each detect an RSSI from the local wireless module 1612. The computing module may determine a first distance from the connectivity module 708 to the local wireless module 1612 and a second distance from the connectivity module 716 to the local wireless module 1612. The computing module may then determine or estimate a location of the local wireless module 1612, 1614 by determining the locations at which a point is the first distance from the connectivity module 708 and the second distance from the connectivity module 712. When a wireless module 1612, 1614 connects to only two connectivity modules 708, 712, 716, the location estimation may include two possible solutions (e.g., one on each side of a line drawn from the first connectivity module to the second). For example, if a local wireless module 1612 is determined to be fifty feet from the connectivity module 708 and sixty feet from the connectivity module 716 to the local wireless module 1612, the computing module may determine that the local wireless module 1612 is located at one of two intersections of a circle with a radius of fifty feet centered on the connectivity module 708 and circle with a radius of sixty feet centered on the connectivity module 716. When a local wireless module 1612, 1614 connects to three or more connectivity modules 708, 712, 716, the location estimation may only include one solution (e.g., the intersection of three circles centered around three connectivity modules 708, 712, 716). In some cases, the RSSI at each connectivity module 708, 712, 716 may be affected by physical obstructions and/or the orientation of the local wireless module 1612, 1614 and the local wireless transceivers. The location estimation may therefore include an area or radius of possible locations that the local wireless module 1612, 1614 could be, rather than a single point, to account for the imprecision of the technology. The locations of any devices with low-energy local wireless modules 1612, 1614 may be visualized on a map alongside the locations of the machines 702, 703, 706, as shown in FIG. 18, described below.

In some embodiments, the computing module may manage a database of devices and machines connected to the local fleet connectivity system, or a plurality of local fleet connectivity systems. The database may be stored, for example, in the cloud 722. A user may access the database via a user device to monitor which of the machines and devices are present at a jobsite. The computing module may receive an indication from a connectivity module 708, 712, 716 that its local wireless transceiver has detected a signal from a local wireless module 1612, 1614 of a device. The computing module may then update the database to associate the device with the local fleet connectivity system. When a device initially connects to a local fleet connectivity system after previously having been disconnected, the computing module may send a notification to a user device that the device has been detected at the jobsite and is connected to the local fleet connectivity system. For example, the computing module may determine that the local wireless transceiver has begun detecting a signal from a local wireless module 1612, 1614 that was not previously detected. The computing module may send the notification to the user device in response to determining that the local wireless transceiver has begun detecting the signal. When a signal from a local wireless module 1612, 1614 stops being detected by any of the local wireless transceivers, the computing module may update the database to disassociate the first device with the local fleet connectivity system and/or send a notification to a user device that the device is no longer detected at the jobsite and the local wireless module 1612, 1614 has recently been removed from the local fleet connectivity system. This can help prevent theft and mistaken transfers of equipment, as the user may be notified when a device is unexpectedly moved off the jobsite.

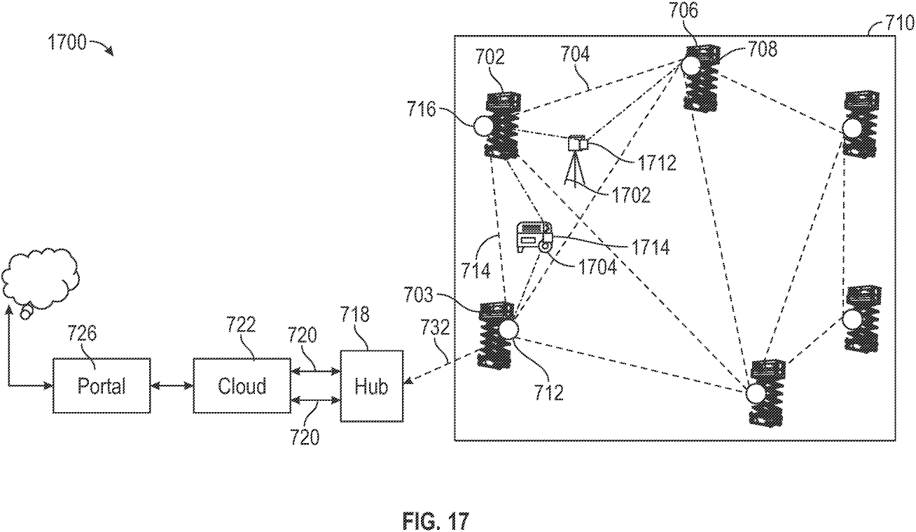

Referring now to FIG. 17, a local fleet connectivity system 1700 is shown, according to some embodiments. The local fleet connectivity system 1700 may be substantially similar to the local fleet connectivity systems 700 and 1600, with the addition of the additional devices 1702 and 1704, respectively shown as a total station and a portable generator. The devices 1702, 1704 may respectively include local wireless modules 1712, 1714, as well as controllers configured to control operation of the devices 1702, 1704. The controllers may be communicably coupled to the local wireless module 1712, 1714, such that the local wireless modules 1712, 1714 are configured to receive device information from the respective controllers and to transmit the device information to the local wireless transceivers of the connectivity modules 708, 712, 716. For example, the controller of the portable generator 1704 may be communicably coupled to a fuel level sensor. The controller may receive the current fuel level of the portable generator 1704 and transmit the fuel level to the connectivity module 708, 712, 716 via the local wireless module 1714, which may then transmit the fuel level to the cloud 722. A user may access the fuel level information via a user device to monitor the fuel level and schedule refueling. Similarly, a controller of the total station 1702 may receive a battery charge level from a battery pack and may transmit the battery charge level to the connectivity module 708, 712, 716 via the local wireless module 1712. The local wireless modules 1712, 1715 may also transmit an activation status (e.g., whether the device is on or off), a GPS location, an energy draw (e.g., the rate at which the device is consuming electricity or fuel), malfunction information, or any other device information available to the controllers. The local wireless modules 1712, 1714 may be more sophisticated than simple RFID tags or BLE tags, as they are able to communicate with the device controllers and may receive and send larger amounts of information. However, the local wireless modules 1712, 1714 may still not be configured to communicate directly with an external computer network, such as the cloud 722, without using one of the connectivity modules 708, 712, 716 as a gateway.

In some embodiments, control instructions may be sent to the controller of a device via the local wireless module 1712, 1714, and the controller may control the device based on the control instructions. Thus, a user may remotely control a device 1702, 1704 by sending control instructions from a user device. By way of example, a user may access, via a user device, a database (e.g., stored in the cloud 722) containing information from machines and devices in the local network of the local fleet connectivity system 1700. The controller of the portable generator 1704 may continuously or periodically send an activation status via the local wireless module 1714 to the connectivity module 708, 712, 716. The connectivity module 708, 712, 716 may then relay the activation status to the cloud 722, where the database can be updated. A user may access the database via a user device and see that the portable generator 1704 is currently turned on and consuming fuel. If, for example, it is currently a holiday and the jobsite is closed, the user may send an instruction to deactivate the portable generator 1704. For example, the user may send the instruction from the user device to the cloud 722, the cloud 722 may then relay the instruction to the connectivity module 708, 712, 716, and the connectivity module 708, 712, 716 may relay the instruction to the local wireless module 1714. The controller of the portable generator 1704 may then receive the instruction from the local wireless module 1714 and deactivate the portable generator 1704. Thus, the local wireless modules 1712, 1714 may operate in substantially the same manner as the connectivity modules 708, 712, 716, except that they may not be configured to directly communicate with external networks like the internet or the cloud 722 without a local gateway, such as the connectivity modules 708, 712, 716. Any controllable feature of the device can be controlled by the user device using these methods.

Referring now to FIG. 18, a user interface 2300 is shown on the screen of a user device 2304, according to some embodiments. The user 2302 may interact with the user interface 2300, for example, by touching the screen of the user device 2304 with one or more fingers 2318. The user interface 2300 may include a map 2308 of a jobsite that indicates the locations of machines and devices connected to a local fleet connectivity system. For example, the map 2308 indicates the location of a plurality of scissor lifts 2310, a plurality of boom lifts 2306, and the portable generator 1704. The map 2308 may indicate which of the machines 2310, 2306 that the portable generator 1704 connects to in order to connect to the local fleet connectivity system. The user interface 2300 may include a list 2312 of the machines and devices, each indicated by an identification number 2314 and accompanied by information regarding the machine or device, such as battery or fuel level, activation status, or malfunction information. The user interface 2300 may also include selectable buttons 2316 that allow the user 2302 to interact with the user interface 2300 or send instructions to the machines or devices. For example, the user 2302 can select a “Locate” button associated with a machine or device that causes the user interface 2300 to focus the map 2308 on the selected machine or device and/or highlight or emphasize the machine or device on the map 2308. The user 2302 can select an “Identify” button causing the associated machine to generate a visual or audible signal so that the user 2302 can find the machine or device on the jobsite. The user 2302 can select a “Power On/Off” button to activate or deactivate the associated machine or device. The user interface may be generated by a software application operating, for example, on the user device or in the cloud. The application may access a database as described above and generate the GUI with the information from the database. The application may enable communication between the user device 2304 and the internet and/or the cloud, such that the user device 2304 can receive data from and send instructions to the machines and devices in the local fleet connectivity system.

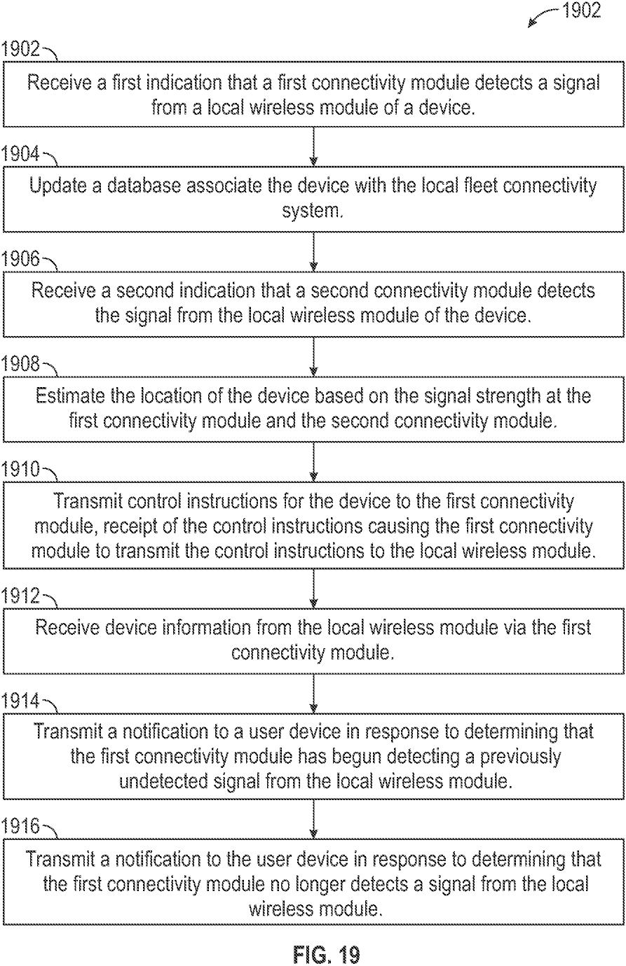

Referring to FIG. 19, a process 1900 (or method) of managing a local fleet connectivity system is shown, according to some embodiments. The operations of process 1900 may be similar or equivalent to the functions of the computing module discussed above with respect to FIG. 17. The method may be performed by one or more processing circuits comprising one or more memory devices (e.g., non-transitory computer-readable storage media) coupled to one or more processors. The one or more memory devices may be configured to store instructions thereon that, when executed by the one or more processors, cause the one or more processors to perform the operations of the method. In some embodiments, the one or more processing circuits may be integrated into a remote computing system (e.g., cloud and web services 268, a computing module as discussed above, etc. In other embodiments, the one or more processing circuits may be integrated into a user device (e.g., user device 272). One or more machines may connect to the user device via a local wireless connectivity system or via a cellular network (e.g., via cell towers 240), or other existing or new means of digital connectivity. The process may include more or fewer operations than shown in FIG. 19, and the operation may be performed in any order, except where specified.

At operation 1902 of the process 1900, a first indication from a first connectivity module of a first machine of a local fleet connectivity system, is received. The first indication indicates that the first connectivity module is within a signal range of and detects a signal from a local wireless module of a device. At operation 1904 of the process 1900, a database is updated to associate the device with the local fleet connectivity system in response to receiving first the indication. The database may identify the machines and devices in multiple local fleet connectivity systems formed by and among the machines and devices. Each local fleet connectivity system may, for example, be associated with a jobsite. The association of a machine or device with the jobsite may indicate that the device or machine is currently located on the jobsite and is either directly connected to the computing module via the external network or is within range with at least one other node (e.g., another machine, a connectivity hub, etc.) configured to connect to the computing module via the external network. The first indication that the first connectivity module detects a signal from a local wireless module of a device may include a first signal strength indication indicating the strength of the signal detected by the first connectivity module. The computing module may determine a distance from the first machine to the device based on the first signal strength indication.

At operation 1906 of the process 1900, a second indication from a second connectivity module of a second machine of a local fleet connectivity system, is received, indicating that the second connectivity module is within a signal range of and detects the signal from the local wireless module of the device. The second indication may include a first signal strength indication indicating the strength of the signal detected by the second connectivity module. The computing module may determine a distance from the second machine to the device based on the second signal strength indication. At operation 1908 of the process 1900, a location of the device may be estimated based on the first signal strength indication and the second signal strength indication. The estimation of the location may also be based on a GPS location of the first machine and a GPS location of the second machine.

At operation 1910 of the process 1900, control instructions for the device may be transmitted to the first connectivity module, receipt of the control instructions causing the first connectivity module to transmit the control instructions to the local wireless module. At operation 1912 of the process 1900 device information relating to the device may be received from the local wireless module via the first connectivity module. The device information may include a battery charge level, a fuel level, an activation status, and/or device malfunction information. At operation 1914 of the process 1900, it may be determined that the first connectivity module has begun detecting the signal after previously not detecting the signal. In response to the determination, a notification may be transmitted to a user device that the device has joined the local fleet connectivity system. At operation 1916 of the process 1900, an indication from the second connectivity module may be received, indicating that the first connectivity module no longer detects a signal from first local wireless module. In response to receiving the second indication, a database may be updated to disassociate the device with the local fleet connectivity system, and a notification may be transmitted to a user device indicating that the device has recently been removed from the local fleet connectivity system.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using one or more separate intervening members, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic. For example, circuit A communicably “coupled” to circuit B may signify that the circuit A communicates directly with circuit B (i.e., no intermediary) or communicates indirectly with circuit B (e.g., through one or more intermediaries).

While various circuits with particular functionality are shown in FIGS. 1-3, it should be understood that the controller 44 may include any number of circuits for completing the functions described herein. For example, the activities and functionalities of the control system 60 may be combined in multiple circuits or as a single circuit. Additional circuits with additional functionality may also be included. Further, the controller 44 may further control other activity beyond the scope of the present disclosure.

As mentioned above and in one configuration, the “circuits” of the control system 60 may be implemented in machine-readable medium for execution by various types of processors, such as the processor 52 of FIG. 1. An identified circuit of executable code may, for instance, include one or more physical or logical blocks of computer instructions, which may, for instance, be organized as an object, procedure, or function. Nevertheless, the executables of an identified circuit need not be physically located together but may include disparate instructions stored in different locations which, when joined logically together, form the circuit and achieve the stated purpose for the circuit. Indeed, a circuit of computer readable program code may be a single instruction, or many instructions, and may even be distributed over several different code segments, among different programs, and across several memory devices. Similarly, operational data may be identified and illustrated herein within circuits and may be embodied in any suitable form and organized within any suitable type of data structure. The operational data may be collected as a single data set or may be distributed over different locations including over different storage devices, and may exist, at least partially, merely as electronic signals on a system or network.

While the term “processor” is briefly defined above, the term “processor” and “processing circuit” are meant to be broadly interpreted. In this regard and as mentioned above, the “processor” may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example, the one or more processors may be a remote processor (e.g., a cloud-based processor). Alternatively or additionally, the one or more processors may be internal and/or local to the apparatus. In this regard, a given circuit or components thereof may be disposed locally (e.g., as part of a local server, a local computing system, etc.) or remotely (e.g., as part of a remote server such as a cloud-based server). To that end, a “circuit” as described herein may include components that are distributed across one or more locations.

Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can include RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Although this description may discuss a specific order of method steps, the order of the steps may differ from what is outlined. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps, and decision steps.

References herein to the positions of elements (e.g., “top,” “bottom,” “above,” “below,” “between,” etc.) are merely used to describe the orientation of various elements in the figures. It should be noted that the orientation of various elements may differ according to other exemplary embodiments, and that such variations are intended to be encompassed by the present disclosure.

Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the components described herein may be constructed from any of a wide variety of materials that provide sufficient strength or durability, in any of a wide variety of colors, textures, and combinations. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claims.

What is claimed is:

1. A local fleet connectivity system, comprising:
a plurality of machines, each comprising an implement and a prime mover configured to drive the implement, each machine coupled to a connectivity module configured to wirelessly communicate with connectivity modules of the other machines to form a local network, wherein a first machine of the plurality of machines includes a first connectivity module comprising a first local wireless transceiver, and at least one of the connectivity modules is configured to communicate with a computing module via an external network; and
a first device coupled to a local wireless module, the local wireless module configured to wirelessly communicate with the first local wireless transceiver to connect to the local network, wherein the local wireless module is not configured to directly connect to the external network.

2. The system of claim 1, wherein the local wireless module communicates via BLE or RFID.

3. The system of claim 1, wherein the computing module is configured to determine a distance from the first machine to the first device based on a signal strength indication at the first local wireless transceiver indicating the strength of a signal received from the local wireless module.

4. The system of claim 1, wherein at least one other machine comprises a local wireless transceiver, wherein the computing module is configured to estimate a location of the first device based on a signal strength indication at each of the local wireless transceivers indicating the strength of a signal received from the local wireless module.

5. The system of claim 1, wherein the computing module is configured to:
receive, from the first connectivity module, an indication that the first local wireless transceiver detects a signal from the local wireless module; and
update, in response to receiving the indication, a database to associate the first device with the local fleet connectivity system.

6. The system of claim 5, wherein the computing module is further configured to:
determine that the first local wireless transceiver has begun detecting the signal after previously not detecting the signal; and
send, in response to the determination, a notification to a user device that the local wireless module has recently been detected.

7. The system of claim 5, wherein the computing module is further configured to:
receive, from the first connectivity module, a second indication that the first local wireless transceiver no longer detects a signal from the first local wireless module;
update, in response to receiving the second indication, the database to disassociate the first device with the local fleet connectivity system; and
send, in response to receiving the second indication, a notification to a user device that the local wireless module has recently been removed from the local fleet connectivity system.

8. The system of claim 1, wherein the local wireless module is communicably coupled to a controller of the first device, the local wireless module configured to receive device information from the controller and to transmit the device information to the first local wireless transceiver.

9. The system of claim 8, wherein the device information comprises one or more of a battery charge level, a fuel level, an activation status, or device malfunction information.

10. The system of claim 1, wherein the local wireless module is communicably coupled to a controller of the first device, the controller configured to receive control instructions from the computing module via the first local wireless transceiver and the local wireless module.

11. The system of claim 1, further comprising a connectivity hub, wherein the connectivity modules are configured to communicate with the external network via wireless communication with the connectivity hub.

12. A non-transitory computer-readable storage medium having instructions stored thereon that, upon execution by a processor, cause the processor to:
receive a first indication from a first connectivity module of a first machine of a local fleet connectivity system, the first indication indicating that the first connectivity module detects a signal from a local wireless module of a device; and
update, in response to receiving first the indication, a database to associate the device with the local fleet connectivity system.

13. The medium of claim 12, wherein the first indication comprises a first signal strength indication indicating the strength of the signal detected by the first connectivity module, and wherein the instructions, upon execution by a processor, further cause the processor to determine a distance from the first machine to the device based on the first signal strength indication.

14. The medium of claim 13, wherein the instructions, upon execution by the processor, further cause the processor to:
receive a second indication from a second connectivity module of a second machine of the local fleet connectivity system, the second indication indicating that the second connectivity module detects the signal from the local wireless module, wherein the second indication comprises a second signal strength indication indicating the strength of the signal detected by the second connectivity module; and
estimate, based on the first signal strength indication and the second signal strength indication, a location of the device.

15. The medium of claim 14, wherein estimating the location of the device is further based on a GPS location of the first machine and a GPS location of the second machine.

16. The medium of claim 12, wherein the instructions, upon execution by the processor, further cause the processor to:
transmit control instructions for the device to the first connectivity module, receipt of the control instructions causing the first connectivity module to transmit the control instructions to the local wireless module.

17. The medium of claim 12, wherein the instructions, upon execution by the processor, further cause the processor to:
receive, from the local wireless module via the first connectivity module, device information relating to the device.

18. The medium of claim 12, wherein the instructions, upon execution by the processor, further cause the processor to:
determine that the first connectivity module has begun detecting the signal after previously not detecting the signal; and
transmit, in response to the determination, a notification to a user device that the device has joined the local fleet connectivity system.

19. The medium of claim 12, wherein the instructions, upon execution by the processor, further cause the processor to:
receive, from the first connectivity module, a second indication that the first connectivity module no longer detects a signal from first local wireless module;
update, in response to receiving the second indication, the database to disassociate the device with the local fleet connectivity system; and
transmit, in response to receiving the second indication, a notification to a user device that the device has recently been removed from the local fleet connectivity system.

20. A method of enabling communication between a device and a cloud computing system via a work machine, the method comprising:
connecting, by a work machine via a short-range wireless communication protocol, to a device;
connecting, by the work machine via a second wireless communication protocol, to a cloud computing system;
receiving, by the work machine, information from the device via the short-range wireless communication protocol;
transmitting, by the work machine based on receiving the information, the information to the cloud computing system via the second wireless communication protocol.
